(19) United States
(12) Patent Application Publication
(10) Pub. No.: US 2022/0144038 A1
(43) Pub. Date: May 12, 2022

(54) SYSTEMS AND METHODS USEFUL FOR AIR TREATMENT IN A VEHICLE

(71) Applicant: Fillenwarth, Daniel, Madeira Beach, FL (US)

(72) Inventor: Daniel P. Fillenwarth, Madeira Beach, FL (US)

(73) Assignee: Fillenwarth, Daniel, Madeira Beach, FL (US)

(21) Appl. No.: 17/492,167

(22) Filed: Oct. 1, 2021

Related U.S. Application Data
(60) Provisional application No. 63/086,533, filed on Oct. 1, 2020.

(51) Int. Cl. B60H 1/00; B60H 3/00; B60H 3/06; A61L 9/20

(52) U.S. Cl. CPC B60H 1/00007; B60H 3/0071; B60H 3/0633; A61L 2209/14; A61L 2209/16; A61L 2209/12; A61L 2209/15; A61L 9/20

(57) ABSTRACT

Described are vehicles having viral inactivation and/or air filtration systems, kits and methods for retrofitting vehicles for viral inactivation and/or filtration of air within a passenger compartment of the vehicles, and methods for viral inactivation and/or filtration of air within passenger compartments of vehicles. The vehicles can be mass transit vehicles such as busses and the viral inactivation and/or filtration systems can include viral inactivation elements such as UV lamps and/or electrostatic filtration apparatuses mounted within a housing or frame mounted or mountable to a wall of the vehicles.

### REFERENCE TO RELATED APPLICATION

This application claims the benefit of priority of U.S. Provisional Patent Application Ser. No. 63/086,533 filed Oct. 1, 2020, which is hereby incorporated herein by reference in its entirety.

### BACKGROUND

The present disclosure relates generally to filtering and/or disinfecting air within passenger compartments of vehicles. In certain embodiments, the present disclosure relates to viral inactivation systems that utilize a viral inactivation element such as a UV lamp to inactivate virus particles in air circulating in the passenger compartment of a vehicle and/or to air filtration systems that utilize an electrostatic filtration apparatus.

As further background, vehicle passenger cabins or compartments present unique challenges in regard to air quality. The density of occupation is often high. A closed vehicle cabin space and high density of occupation, together with the recirculation of the passenger compartment air provides an environment in which viruses and other germs emitted from sick passengers can be readily transmitted to those who are not yet infected and in which other circulating particulate matter may be harmful or irritating to passengers.

In view of the background in this area, there are needs for improved and/or alternative systems, apparatuses and methods for inactivating pathogens, such as viruses, in the circulating air of a vehicle passenger compartment, and/or for filtering out particulates in such circulating air. Desirably, such systems and methods would be cost effective and convenient in implementation, while nonetheless effective in inactivating pathogens and/or filtering particles.

### BRIEF DESCRIPTION OF THE DRAWINGS

FIG. 1 provides a schematic representation of a viral inactivation system onboard a vehicle in accordance with one embodiment.

FIG. 2 provides a front view of a viral inactivation system housing mounted to a wall of a vehicle in accordance with one embodiment.

FIG. 3 provides a partial cutaway side view of a viral inactivation system housing wherein a side panel of the housing has been removed to show a UV lamp and a portion of a sight-tight grille within the housing.

FIG. 4 shows a front view of a viral inactivation system housing in accordance with an embodiment herein having the front panel thereof in a closed position.

FIG. 5 shows a front view of the viral inactivation system housing of FIG. 6 having the front panel thereof in an open position.

FIG. 6 provides a perspective view of a vehicle in which a viral inactivation system herein has been retrofitted.

FIG. 7 provides a perspective back side view of one embodiment of an air filtration unit herein.

FIG. 8 provides a perspective front side view of the embodiment of FIG. 7.

FIG. 9 provides partial cutaway back side view of the embodiment of FIG. 7 having the filter media and distributor element removed.

FIG. 10 provides a partial cutaway back side view of the embodiment of FIG. 7 having the filter media and distributor element removed and a rearward filter media mount frame pivoted to an open position.

FIG. 11 provides a perspective back side view of an air filtration and disinfection embodiment similar to FIGS. 7 to 10 and additionally having an ultraviolet disinfection source.

FIG. 12 provides a schematic cross-sectional view of a modified filter media arrangement that can be used in disclosed air filtration and/or disinfection embodiments.

FIG. 12A provides a schematic cross-sectional view of another modified filter media arrangement that can be used in disclosed air filtration and/or disinfection embodiments.

FIG. 13 provides a perspective view of another air filtration and disinfection apparatus of the present disclosure.

FIG. 14 provides a perspective view of the apparatus of FIG. 13 with a housing access door in an open position.

FIG. 15 provides a schematic view of another air filtration and disinfection apparatus of the present disclosure installed in a railcar with a roof-mounted HVAC system.

FIG. 16 provides a perspective view of another air filtration and disinfection apparatus of the present disclosure installed in a vehicle (e.g. bus) conjunction with a ceiling-mounted HVAC unit.

FIG. 17 provides a perspective view of the internal components of the apparatus of FIG. 16.

FIG. 18 provides a partial cutaway perspective view of another embodiment of an air filtration and disinfection system fitted on the rear side of an air return grille of a bus.

FIG. 19 provides a back side view of the air return grille of FIG. 18, with a bulb rack in an operating position adjacent an electrostatic filter apparatus.

FIG. 20 provides a view of the air return grille of FIG. 19, with the bulb rack swung on a hinge away from the electrostatic filter apparatus.

FIG. 21 provides a schematic diagram of another embodiment of ani air filtration and disinfection apparatus of the present disclosure installed in the ceiling region of a vehicle (e.g. bus).

### DETAILED DESCRIPTION

Reference will now be made to certain embodiments and specific language will be used to describe the same. It will nevertheless be understood that no limitation of the scope of the disclosure is thereby intended. Any alterations and further modifications in the described embodiments and any further applications of the principles as described herein are contemplated as would normally occur to one skilled in the art to which this disclosure relates.

As disclosed above, certain aspects of the present disclosure relate to vehicles having viral inactivation systems, kits and methods useful for retrofitting vehicles for viral inactivation of air within a passenger compartment of the vehicles, and methods for viral inactivation of air including within passenger compartments of vehicles.

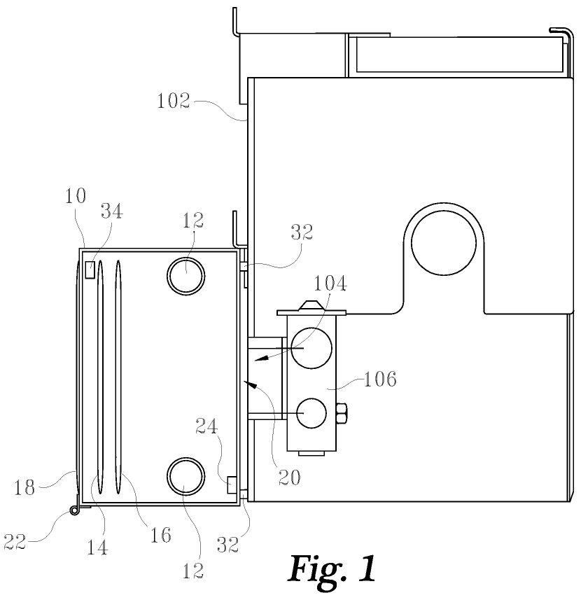

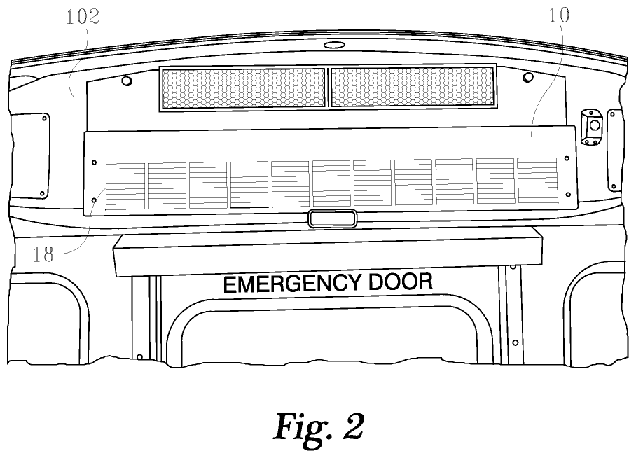

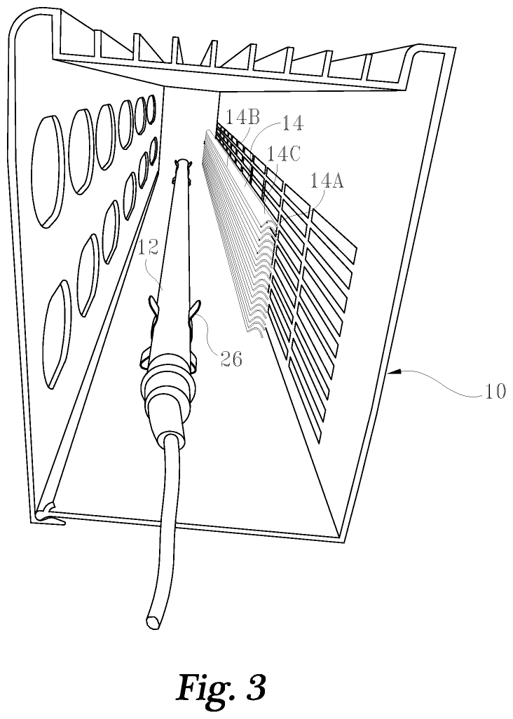

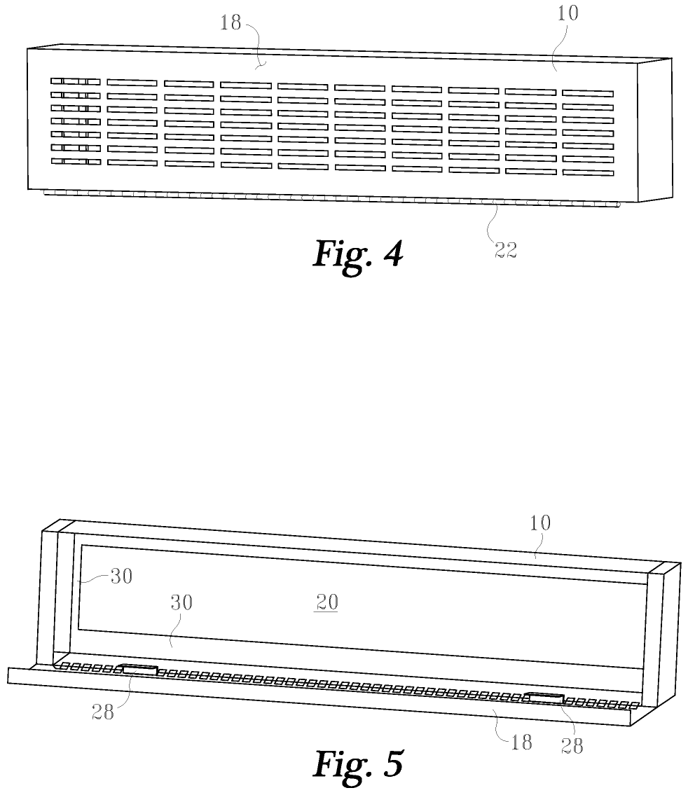

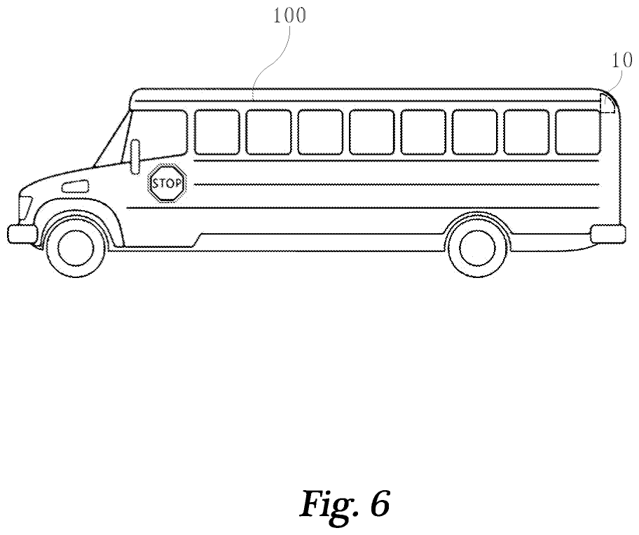

With reference now to FIGS. 1 through 6, illustrated is one embodiment of a viral inactivation system, and a vehicle incorporating the same. The vehicle 100 is preferably one having an interior passenger compartment with seating for many human occupants, for example at least 10 occupants, or at least 20 occupants, or at least 30 occupants, and typically in the range of about 20 to 100 occupants. In the specific illustrated embodiment the vehicle is a bus, such as a school bus. The viral inactivation system includes a housing 10 mounted to a wall, such as a back wall 102, of a bus 100, and in the illustrated embodiment extending into the passenger compartment from the wall. The housing 10 has an interior space that houses at least one ultraviolet light source 12, such as an ultraviolet (UV) lamp or bulb, and in some embodiments two, or two or more, such ultraviolet light sources. The viral inactivation system also includes an air intake grille 14 connected to the housing 10. The air intake grille 14 can prevent occupants within the vehicle interior passenger compartment from being exposed to UV rays contained in the UV irradiation field within the housing 10. The grille 14 may limit or prevent direct viewing of the UV rays emitted from the source(s) by occupants of the interior passenger compartment of the vehicle 100, and in some forms can also prevent reflections of the UV rays from reaching the occupants. For example, the grille 14 and its associated housing 10 may limit an occupant's exposure (including eye exposure) to the UV rays to below threshold limit values, as are well known in the art, so as to avoid harm to the occupant. Nonetheless, the grille 14 allows the passage of air through the grille 14. In some embodiments, the grille can include a plurality of stacked grille members that include a first segment 14A extending in the first direction and a second segment 14B extending in a second direction angled from (not parallel with) the first direction, with a peak 14B adjoining the first segment 14A and the second segment 14B. In this manner, a convoluted flow path is created in between adjacent ones of the grille elements so as to allow the passage of air, but also so as to prevent the passage of direct UV rays between the adjacent grille elements. A so-called “sight-tight” grille can suitably be used for these purposes. The viral inactivation system also includes in some embodiments a filter 16, preferably a washable filter (e.g. an aluminum or other metal filter), within the housing 10. In the illustrated embodiment, the filter 16 is positioned downstream in the air flow direction relative to the air intake grille 14, so that air passing into the housing 10 first contacts grille 14 and then contacts filter 16.

The housing 10 has a front wall or panel 18 having an opening or a plurality of openings, for example providing a grille in the front wall 18. The back wall or panel of housing 10 includes an opening 20 to be aligned with the opening 104 in the back wall 102 of the vehicle 100 through which air flows to enter the air conditioning system. In the illustrated embodiment, the front wall 18 of the housing 10 is connected by a hinge 22 to the housing 10, for example a so-called piano hinge, so that the front wall 18 can be hingedly opened to access the interior space of the housing 10. The front wall 18 can be held securely in the closed position by a latch, screw or other suitable mechanism, which can be operated or removed to hingedly open the front wall 18.

The illustrated system also includes an inverter 24, which can be housed within or otherwise attached to the housing 10, for converting direct current to alternating current. In this manner, where the vehicle 100 is equipped to provide direct current, for example generated using an alternator associated with an engine of the vehicle 100 and/or in the case of an electrically driven vehicle via batteries that drive the vehicle 100, and where the ultraviolent light source or sources 12 are configured to operate on alternating current, the inverter 24 can convert the direct current provided by the vehicle 100 to alternating current that can be used to power the ultraviolent lights source or sources 12. In other forms, the ultraviolet light source or sources 12 can be configured to operate on direct current, and the use of the inverter can be avoided, e.g. while using a converter if needed to alter the voltage fed to the source(s) 12. The viral inactivation system can be electrically connected to an electrical power source in any suitable manner, and can include a power feed cord(s) and/or wire(s) for achieving such a connection, e.g. in a kit for retrofitting a vehicle with the system. A switch for turning on and off the power to the source or source(s) 12 can be provided at any suitable location in the vehicle 100, including in the vicinity of the housing 10 and/or in the vicinity of a driver area of the vehicle 100. As well, in a preferred form, a switch 34 (e.g. an interlock switch) is associated with the front panel 18 of housing and is configured in the power feed to source(s) so as to interrupt (turn off) power to the source(s) when the panel 18 is opened.

The housing 10 also includes at least one and in some forms a plurality of clips 26 or other mount elements so as to stably mount the ultraviolent light source or sources 12 within the housing 10. In some forms, these mount elements can have or be associated with a vibration damping material or element (for example a spring), as is known in the art. In preferred forms, housing 10 also includes mounting elements 28, for example grooved clips, for removably mounting the air intake grille 14 and the filter 16 within or otherwise to the housing 10. In this regard, it is contemplated that the grille 14 and filter 16 can be stacked and mounted together so as to share the mounting element or elements 28 (for example having the grill/filter stacked construction held by its periphery into grooves of clips 28 or other mounting elements), but in other forms the grille 14 and the filter 16 can each have their own mounting element or elements for mounting in or to the housing 10.

In some forms, at least the panels or walls of housing 10 can be made of metal, such as painted steel. In addition or alternatively, the interior surfaces of such panels or walls can be reflective so as to enhance the efficiency of disinfection by the UV light source(s) within the housing 10. For example, when a reflective metal such as steel is used to make the walls or panels of housing 10, an interior surface of the panels can be left unpainted and reflective, while exterior surfaces of the panels can if desired be painted to enhance the external appearance of the housing 10. The housing 10 in some forms will have an interior space having a volume of at least 500 cubic inches, for example in the range of about 500 to about 3000 cubic inches, and preferably in the range of about 1000 to about 1700 cubic inches. In addition or alternatively, the housing 10 and the UV light sources(s) can be sized and positioned relative to one another such that no inner surface of the housing 10 (in its closed condition) is more than a specified distance from at least one UV light-emitting surface of a UV lamp or other UV light source, where this specified distance in some forms is 6 inches, preferably 4 inches, and more preferably 3 inches. In this manner, air flowing through the housing will necessarily pass close to the UV light source(s), thereby facilitating the system's efficacy.

The bus or other vehicle 100 will typically include an existing air conditioning system with elements for heating and/or cooling air circulated within the interior passenger compartment of the vehicle 100, including a fan or fans for powering air flow in a path from the interior passenger compartment, through heating and/or cooling elements of the air conditioning system, and then back into the interior passenger compartment of the vehicle 100. Illustratively, as shown particularly in FIG. 1, an air conditioning system can include a coil 106 against which a circulating air is passed to cool the air. In the illustrated embodiment, the coil 106 and at least some of the other elements of the air conditioning system of the vehicle 100 are mounted behind the back wall 102 of the vehicle 100 bounding the interior passenger compartment. Also in the illustrated embodiment, the back wall 102 has an opening 108 spaced from the opening 104, and the conditioned air returns to the interior passenger compartment of the vehicle 100 through the opening 108. In the illustrated embodiment, the opening 108 occurs above the opening 104.

In some embodiments herein, the viral inactivation system is retrofitted onto the vehicle 100 having the existing air conditioning system. For example, an existing grille covering opening 104 in wall 102 of the vehicle 100 can be removed. Then the housing 10 and other above-described components of the viral inactivation system attached to and/or housed within housing 10, which can in some embodiments be provided in a retrofitting kit, can be mounted to the back wall 102. This mounting can be conducted in any suitable manner. In some forms, screws or other connectors can be inserted into the back wall 102, for example through flanges 30 surrounding opening 20 of the back wall of the housing 10, so as to mount the housing 10 to the back wall 102. Also in some forms, a sealing element can be positioned between the back wall or panel of housing 10 and the back wall 102 of the vehicle 100, so as to resist or prevent airflow occurring from the back periphery of housing 10 into spaces between the back wall of housing 10 and the back wall 102 of vehicle 100. Compressible materials such as compressible foam materials located around the outer periphery of the back wall of housing 10, e.g. associated with the rear surface of flanges 30, can be suitably used for these purposes.

In use, the air conditioning system of vehicle 100 can be operated as to draw air from the interior passenger compartment through the opening or openings in front panel 18 of housing 10, through the grille 14, through the filter 16, and passed the ultraviolent light source or sources 12. The air is treated by the ultraviolent light sources 12 so as to inactive virus particles, for example coronavirus viral particles such as SARS-CoV-1 and/or SARS-CoV-2 particles. The virally inactivated air then passes through opening 104 of rear wall 102, and then through elements of the air conditioning system, and through opening 108 to return to the interior passenger compartment of vehicle 100.

In some embodiments, the vehicle 100 will have only a single air conditioning source, for example in the back wall of the vehicle as in the illustrated embodiment. In other forms, the vehicle 100 can have multiple air conditioning sources and any one or some of, or each, may be equipped with the housing 10 and other elements of the viral inactivation system disclosed herein. For example, some vehicles such as some buses have an air conditioning source such as that disclosed in the Figures also in the front wall of the bus or other vehicle. It will be understood that an arrangement similar to that disclosed in FIG. 1 and the other Figures herein can be provided both at the back source and the front source of air conditioning of the vehicle in further embodiments herein.

According to some embodiments, the ultraviolet light source or sources 12 can be UV-C emitter(s) that irradiate in the range between 200-280 nanometers producing UV rays that destroy the DNA or other nucleic acid molecules of the virus particles. The UV radiation can also destroy or inactivate other harmful microorganisms such as bacteria or mold spores. Advantageously when used, the UV-C emitter(s) can have a peak emission at about 254 nanometer and/or can irradiate UV at an intensity of at least about 200 microwatts/cm² at 1 meter, for example in the range of about 200 to about 300 microwatts/cm² at 1 meter. The UV lamp(s) are preferably non-ozone producing lamp(s) that produce short-wave ultraviolet energy. Additionally, a glass envelope of the UV-C lamp can be transparent to the UV-C wavelengths and doped with ozone-suppressing agents. Relatively elongate ultraviolet light sources, such as ultraviolet lamps, will be beneficially used, for example having a length of at least about 12 inches, or at least about 24 inches, or at least about 36 inches, and typically in the range of about 12 inches to about 48 inches.

The air conditioning system or systems of the vehicle of this and other embodiments disclosed herein can be configured to change the air within the interior passenger compartment at a rate of at least about 30 air changes per hour, or at least about 50 air changes per hour, for example in the range of about 30 to about 150 changes per hour or about 50 to about 150 changes per hour, more preferably in some forms about 100 to 150 air changes per hour. This relatively high air-processing rate aids in achieving effective viral inactivation of the air circulating within the interior passenger compartment in embodiments herein. In addition or alternatively, the air conditioning system and the viral inactivation system herein can be configured to achieve and air flow rate through the housing 10 of at least about 1000 feet per minute, for example in the range of about 1000 to 1500 feet per minute. Again, at high flow rates such as these, the high air change rates per hour provided in preferred embodiments aids in achieving effective viral inactivation of the air within the interior passenger compartment. As those skilled in the field will understand, such air change and flow features will involve many passages of the air through the housing and its interior ultraviolet light field and thereby can provide high levels of viral inactivation for the overall passenger compartment.

The direction of air flow relative to the longitudinal axis of an ultraviolet light source or source(s) can be selected as appropriate to achieve inactivation of the viral particles. In the illustrated embodiment, the direction of air flow is generally perpendicular to the longitudinal axis of elongate ultraviolet light source(s) 12. It is also contemplated that the direction of air flow can be generally parallel to the longitudinal axis of the source(s) 12. In some such forms, the housing 10 can be provided with an opening or openings in one or both of its side panels (rather than in its front panel), so that the negative pressure provided at opening 104 of wall 102 by the air conditioning system draws air through the side panel(s) to travel a distance generally parallel to the longitudinal axis of the source(s) before entering the opening 104. Angles between parallel and perpendicular to the longitudinal axis of the source(s) 12 may also be employed, as can internal baffles in the housing 10 if desired to increase the residence time of air passing through the housing in the UV field created by the source(s) 12.

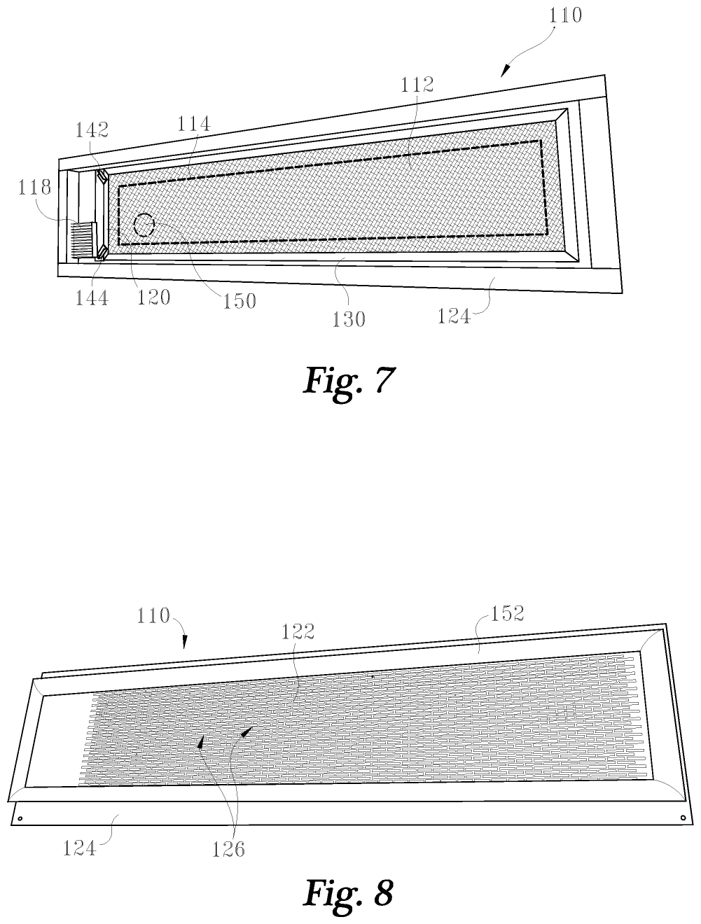

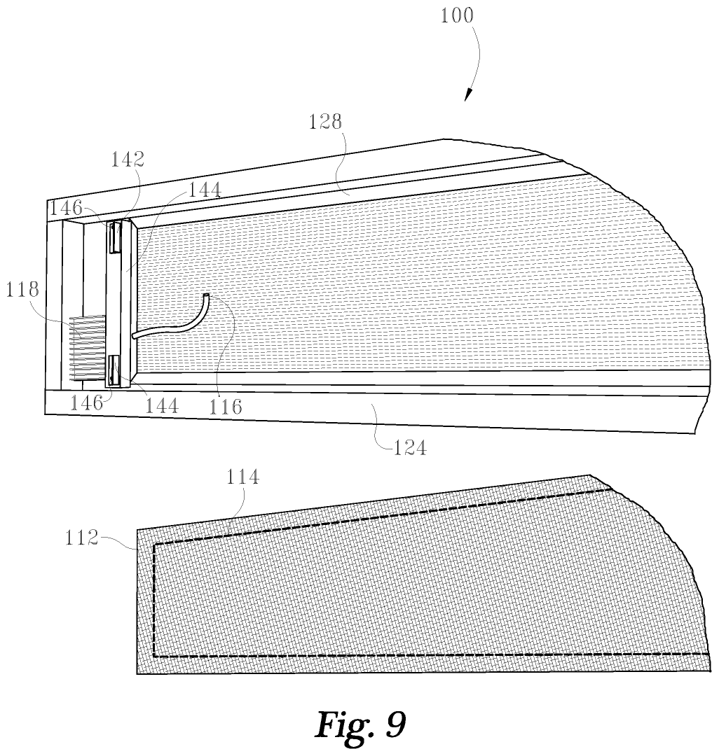

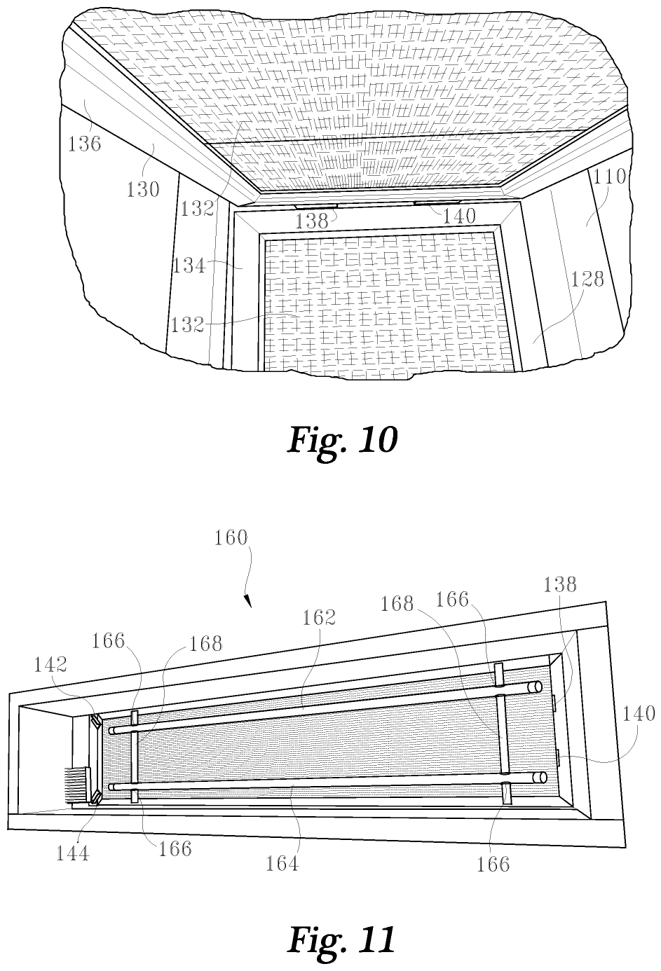

With reference now to FIGS. 7 to 10, illustrated is an embodiment of a high efficiency filter unit 110 that can be installed in a vehicle to filter air in a passenger compartment of a vehicle, for example any vehicle as disclosed herein. Filter unit 110 incorporates an electrostatic filtration apparatus 111 that includes a fibrous dielectric filter media 112, for example a fiberglass filter media, and a conductive distributor element 114 such as a screen or mesh, for example made from activated carbon, contacting the filter media 112. The fibrous dielectric filter media 112 can be provided by one or more layers or plies (e.g. two-ply) and can have a total thickness in the range of about 0.25 to about 2 inches, or 0.5 to 1.5 inches. The distributor element 114 can be enclosed between plies of the filter media 112 and can be replaceable or disposable with the filter media 112. A voltage is applied to distributor element 114 by a contact probe 116 to create an electrostatic field and polarize the filter media 112. An appropriate electrical power source energizes contact probe 116. The power source can be an electrical power source of a vehicle in which filter unit 110 is installed, and can be a direct current DC power source. In some embodiments, the filter unit includes a voltage converter 118 which can for example convert the voltage (e.g. 12V) of a vehicle's power source to a higher voltage (e.g. 24V), for example when connected to the vehicle's power source by lead wires to the converter (shown e.g. in FIG. 9). The filter unit 110 can also include a power supply 120 for the contact probe 116, with the power supply operable to convert the output of the converter 118 to a voltage at which the contact probe 116 is configured to operate. The contact probe 116 in embodiments herein can be configured to operate at a high voltage, for example at least 5000 volts, and typically in the range of about 5000 to about 8000 volts, so as to polarize filter media 112 through the action of distributor element 114. The polarized filter media 112 electrostatically attracts particles to be filtered from a gaseous flow through the filter unit 110, for example the air flow from a passenger compartment of a vehicle in which filter unit 110 is installed. A suitable electrostatic filtration apparatus is available from Dynamic Air Quality Solutions of Princeton, N.J., USA, and can be modified if and as necessary to provide the features described herein.

In preferred forms, the filter unit 110 can be provided as a convenient apparatus for installation and use in a vehicle. To this end, the filter unit 110 can include a front (passenger compartment-facing) grille 122 through which the air passes before contacting filter media 112. Filter unit 110 can also include a mount means such as a housing 124 attached to grille 122 and configured for mounting to a vehicle, for example having openings 126 for receiving connectors (e.g. screws, bolts, rivets, etc) to mount unit 110 to the vehicle, for example a wall of the vehicle bounding the passenger compartment. The mount means can also be a frame or rack in other forms. The filter media 112, distributor element 114, contact probe 116, converter 118 and/or power supply 120 can be held to the housing 124, for example mounted within the housing 124. In some forms, filter media 112 is received between a first frame element 128 and a second frame element 130, with the frame elements 128, 130 being electrically grounded and constructed and arranged to permit flow of air through them. Illustratively, frame elements 128 and 130 can include a screen material 132, for example comprised of a metal material (e.g. aluminum) defining a plurality of openings. Frame elements 128 and 130 can also include peripheral frames 134 and 136, for example made of aluminum, to which screen material 132 is mounted at its peripheral edges. Frame elements 128 and 130 can be attached to one another, for example so as to allow partial or complete separation of elements 128 and 130 for removal and replacement of filter media 112 potentially along with distributor element 114, followed by repositioning elements 128 and 130 to sandwich the filter media 112 and distributor element 114. In some forms, frame elements 128 and 130 are hingedly attached to one another along an edge, for example with hinges 138 and 140. In such forms, a separating pivotal motion can be used to separate frame elements 128 and 130 for filter media 112 (potentially also distributor 114) replacement, and a closing pivotal motion can be used to sandwich the newly installed material(s) between frame elements 128 and 130. For example, frame element 128 nearest the grille 122 can be fixed in position relative to the housing 124 (e.g. by attachment to the housing 124 and/or grille 122), and frame element 130 can be free to pivot relative to the housing 124. In such embodiments, frame element 130 can be pivoted away from and thereafter back toward frame element 128 for media 112 and/or distributor element 114 replacement or other operations. To hold the frame elements 128 and 130 in position sandwiching the filter media 112, filter unit 110 can include one or more releasable stops with a released position allowing separation of the frame elements 128 and 130 from one another (e.g. by pivotal motion) and a secured position preventing separation of the frame elements 128 and 130 from one another. For example, filter unit 110 can include at least first and second releasable stops 142 and 144 configured to contact frame element 130 in a secured position and thereby prevent its separation from frame element 128, with the stops 142 and 144 movable out of contact with frame element 130 to a released position to allow separation of frame element 130 from frame element 128. Releasable stops 142 and 144 may, for example, be rotatable tabs that are rotatably mounted at or near one end by a rivet 146 or other connector to the housing 124, for example a cross-bar 148 of the housing 124 positioned adjacent a corresponding edge of the frame element 130 (e.g. edge of mount frame 136).

In certain variants, filter unit 110 also includes a protective element 150 such as a cap positioned against filter media 112 opposite the contact probe 116. Protective element 150 can be comprised of a suitable plastic or metal material. Protective element 150 can be mounted to frame element 130 and positioned to align with the tip of contact probe 114 when frame element 130 is in position sandwiching the filter media 112. Additionally or alternatively, embodiments of filter unit 110 can include polymeric foam padding material 152 mounted to portions of housing 124 and/or grille 122 that will be exposed to the passenger compartment of the vehicle.

Filter unit 110 can be installed as an original manufacture item or as a retrofit at any suitable location, for example in a wall bounding the passenger compartment of a vehicle, for example a bus, a train car, an automobile, or another vehicle. The electrical power to the filter unit 110 can be drawn from the electrical system of the vehicle in any suitable fashion, and in retrofit applications can be provided by an existing electrical power wire, e.g. proximate to the retrofit installation location for filter unit 110. In some retrofit cases, filter unit 110 will replace or supplement an existing air return grille of the vehicle and thus will employ an existing opening in a wall bounding the passenger compartment, e.g. with or without resizing the opening (including using the opening as is or potentially enlarging or decreasing the size of the opening). In other cases, a new air return opening will be created in such a wall and the filter unit 110 will be mounted and deployed in association with the new air return opening.

The electrostatic filter apparatus 111 of filter unit 110 can provide at least a Minimum Efficiency Reporting Values equivalent rating of 13 (MERV-13 equivalent), or greater, for example MERV-13, MERV-14, MERV-15, or MERV-16 equivalent, as determined by the well-known test method of the American Society of Heating, Refrigerating, and Air Conditioning Engineers (ASHRAE) 52.2 Appendix J. Additionally or alternatively, the filter unit 110 can exhibit a capacity to remove about 50% to about 99% of particles in the size range of 0.3 microns to 1.0 microns. The filter apparatus can, for example, be effective to capture aerosols that contain viral particles on the filter media 112. The filter unit 110 can be configured to operate practicably within vehicle installations in which air flow through the unit 110 is powered by a fan operating at a practicable power draw for a vehicle, for example no greater than about 40 amps and in some cases in the range of about 10 to about 40 amps. Using one or more such fans, for example one, two or three such fans, high levels of air exchange in the passenger compartment can be provided, for example as specified herein. In some embodiments, this low-load efficiency is provided despite their being one or multiple (e.g. 2) additional filters downstream of the unit 110 in the air circulation system of the vehicle also causing load on the fan(s). Filter unit 110 can be deployed in association with air intakes for air circulation systems that include air conditioning (heating and/or cooling) or air circulation systems without air conditioning.

With reference now to FIG. 11, illustrated is an embodiment of an air disinfection unit 160 of the present disclosure. Air disinfection unit 160 has many components and functions that correspond to those of air filter unit 140 of FIGS. 7 to 10, with some of those components being shown and similarly numbered in FIG. 11. Air disinfection unit 160 adds to those components one or more ultraviolet light sources for disinfection (e.g. viral inactivation) of the air. In the embodiment of FIG. 11, unit 160 includes a first ultraviolet lamp 162 and a second ultraviolet lamp 164. Lamps 162 and 164 are arranged to receive air flow after passage through filter media 112. Lamps 162 and 164 thereby disinfect the air by application of ultraviolet radiation as describe herein, for example effective to inactivate pathogens such as viral particles and/or bacteria. In the illustrated embodiment, lamps 162 and 164 are mounted in a plurality of clips 166 or other mount elements so as to stably mount the lamps in the unit 160. In some forms, these mount elements can have or be associated with a vibration damping material or element (for example a spring), as is known in the art. The mount clips 166 are in turn mounted to support bars 168 attached to the frame element 130 (e.g. mount frame 136). In this manner, a pivotal motion of frame element 130 to replace or otherwise access filter media 112 also pivots lamps 162 and 164 out of the way to clear an access path to filter media 112. Lamps 162 and 164 can also be powered by an electrical system of a vehicle in which the unit 160 is installed, either as a retrofit or during original manufacture of the vehicle.

While the embodiment illustrated in FIG. 11 includes the ultraviolet radiation source (lamps 162 and 164) integrally associated in a unit with an electrostatic filtration apparatus 111, in other embodiments the ultraviolet radiation source (e.g. one or more UV lamps) can be separately installed, preferably downstream of the electrostatic filtration apparatus 111 so that the air first passes through the filtration apparatus 111 and then is disinfected by the ultraviolet radiation source. In certain embodiments, one or more UV lamps can be separately installed in a return air duct of a vehicle that receives air after passage through the electrostatic filtration unit. These and other variations will be apparent to those skilled in the field from the descriptions herein.

As discussed above, the fibrous dielectric filter media 112 can be provided by one or more layers (e.g. pleated layers) and//or can have a total thickness in the range of about 0.25 inches to about 2 inches, or about 0.5 inches to about 1.5 inches. The media 112 is preferably formed from a material, for example glass, that is substantially permeable to the disinfectant UV light provided by the source(s) 162 and/or 164. This aids in the inactivation of viral particles and/or other pathogens that are caught on media 112 by the ultraviolet light source(s) 162 and/or 164. In beneficial embodiments herein, the ultraviolet light source(s) and the fibrous dielectric filter media 112 will be configured such that ultraviolet light at an intensity of at least about 1 milliwatt/cm², or at least about 5 milliwatts/cm², and in some forms in the range of about 1 milliwatt/cm² to about 40 milliwatts/cm², or about 3 milliwatts/cm² to about 20 milliwatts/cm², exits the surface of filter media 112 that is positioned opposite of the ultraviolet light source(s) 162 and/or 164. Such embodiments provide preferred penetration of the ultraviolet light and inactivation of viral particles or other pathogens caught on the filter media 112.

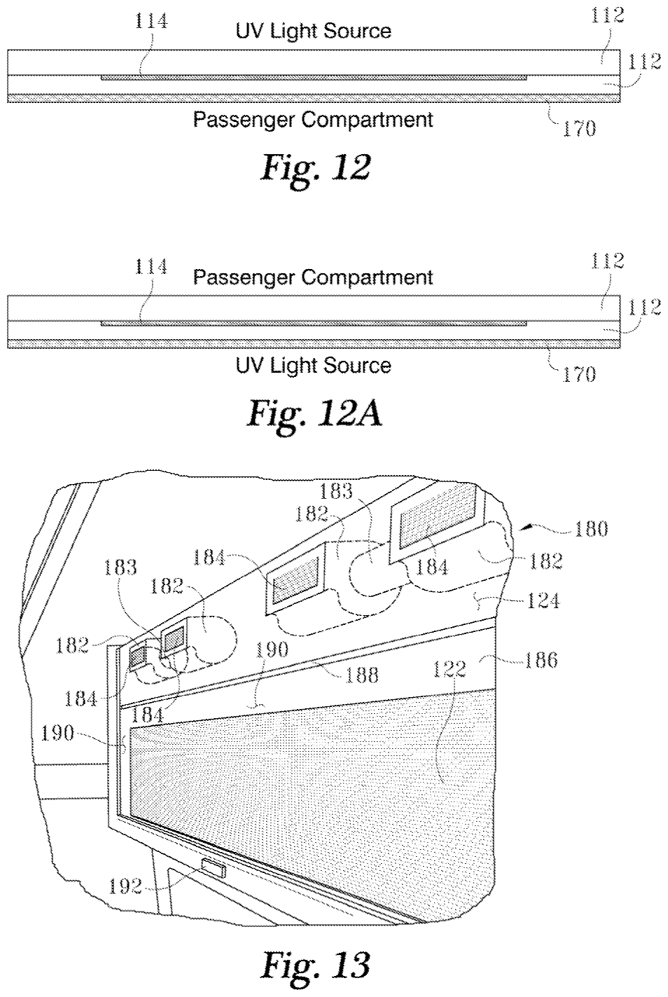

In some forms, the ultraviolet radiation source, whether integral with the electrostatic filtration apparatus and separately installed, will deliver ultraviolet radiation to the filter media 112 sufficient to inactivate pathogens, for example viral particles or bacteria, that are caught on the filter media 112 (e.g. either directly or through other filtered solids adhered to the filter media 112). The emission profile of the ultraviolet radiation source, its spacing from the filter media 112, and the permeability of the filter media 112, and other factors can be selected to achieve such pathogen inactivation on the filter media 112, and in preferred forms can provide ultraviolet light intensities at the far (UV-exiting) surface of the filter media 112 as taught hereinabove. In addition or alternatively, the filter media 112 itself and/or its combination with another fibrous material can be effective to prevent the passage of harmful levels of ultraviolet radiation into the passenger compartment of the vehicle. The other or secondary fibrous material can be less penetrable by the emitted ultraviolet radiation than the fibrous dielectric filter media, and is preferably electrically non-conductive. For example, illustrated in FIG. 12 is a schematic cross-sectional view of an arrangement of layers of filter media 112 along with secondary (e.g. carbon) fiber layers 170 that can be used for these purposes. It will be understood that one or more layers of carbon fibers and/or other secondary fibers may be used, depending for example on their thickness and density, to aid in preventing UV passage into the passenger compartment. In preferred forms at least one such supplemental carbon or other fiber layer will be positioned to a side of filter media 112 opposite the ultraviolet radiation source and between the filter media 112 and the passenger compartment. The total thickness of such supplemental fiber layer(s) can, for example, be in the range of about 0.2 inches to 1 inch, or about 0.2 inches to about 0.5 inches. Of course, a sight-tight grille such as that described hereinabove can optionally be used in addition to or as an alternative to such a selection and arrangement of the filter media 112 alone or with supplement carbon fiber or other fiber material to block passage of UV radiation. These and other means for blocking UV radiation can be used. In certain embodiments of UV disinfection or air filtration/UV disinfection apparatuses disclosed herein, the UV intensity at a wavelength of 254 nanometers at the outer surface of the return air grille (e.g. grille 14 or 122) will be less than about 10 μW/cm², or less than about 5 μW/cm², or less than about 3 μW/cm², or less than about 2 μW/cm², or less than about 1 μW/cm². In more preferred such embodiments, the UV intensity at a wavelength of 254 nanometers at the outer surface of the return air grille (e.g. grille 14 or 122) will be less than about 0.5 μW/cm², or less than about 0.1 μW/cm², or less than about 0.01 μW/cm², and in some forms zero. A sight-tight grille and/or a UV-blocking fibrous media arrangement as disclosed herein can serve to reduce what would otherwise be a higher UV intensity at 254 nanometers at the outer surface of the return air grille to a value within these recited ranges. With reference to 12A, in other embodiments, at least one supplemental carbon or other fiber layer 170 can be positioned to a side of filter media 112 facing the ultraviolet radiation source(s). Such an arrangement can be used, for example, where the filter media 112 and/or the distributor element 114 is sensitive to degradation by the emitted ultraviolet radiation.

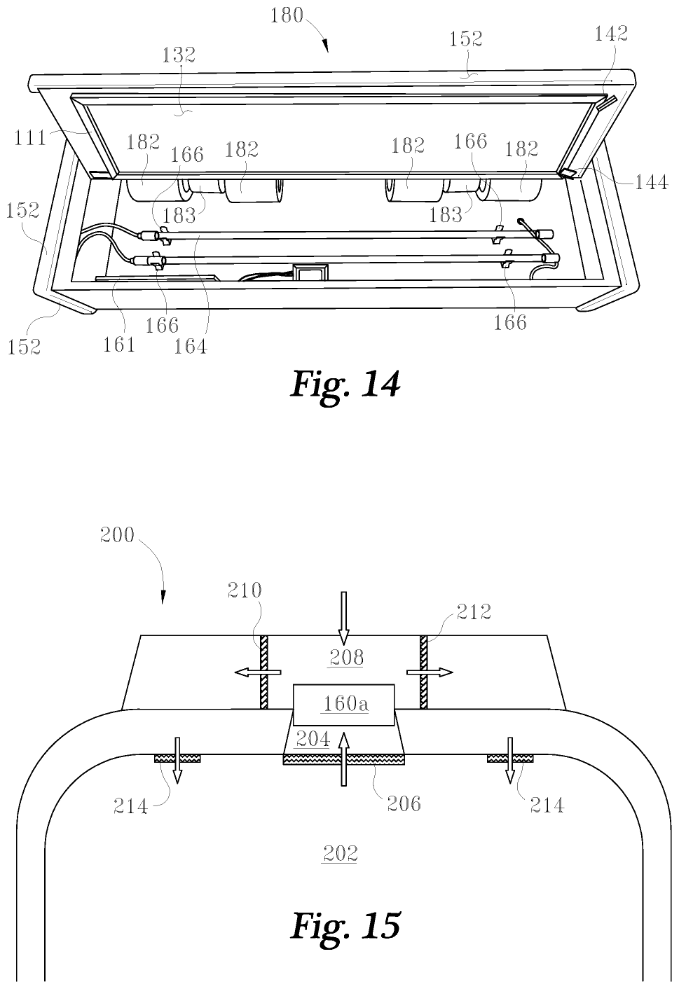

With reference now to FIGS. 13 and 14, illustrated is another embodiment of the present disclosure. Shown is an air filtration and disinfection apparatus 180 that can be installed in a vehicle to treat air within a passenger compartment of the vehicle. Apparatus 180 includes UV source and filtration components similar to those of other embodiments herein, which are similarly numbered in FIGS. 13 and 14. Apparatus 180 further includes at least one fan driven by an electrically powered motor, and preferably a plurality of such fans 182, arranged to draw return air into housing 124 through grille 122, through dielectric filter media 112 and when present through UV-blocking carbon or other fiber layer(s) 170, and through the disinfecting UV field created by the UV lamp(s) (e.g. 162, 164). The thus-treated air travels internally within housing 124 and is then supplied back to the passenger compartment through an opening or openings 184 (which can be covered for example with grille(s) or louver(s)) associated with the fan(s) 182, with the opening(s) 184 at a position on the housing 124 that is spaced from the return air grille 122. Apparatus 180 includes a housing access door 186 hingedly attached to housing 124 through access door hinge 188 such as a piano hinge running the full length or essentially the full length of the door 186. The return air grille 122 is mounted or incorporated in housing access door 186, and in the illustrated embodiment is surrounded by a peripheral solid (non-grille) panel portion 190. Housing access door can include a latch 192 that can be actuated to unlatch door 186 so that it can be opened. Latch 192 can be a child-proof, tamper-proof, or lockable latch.

Referring particularly to FIG. 14, apparatus 180 is shown with housing access door 186 swung to an open position. The electrostatic filtration apparatus 111 is mounted to access door 186 and moves with door 186 when it is opened and closed. In particular, in illustrative apparatus 180, the first filter media frame 128 is fixedly mounted to and positioned inside the door 186, and the second filter media frame 130 is hingedly mounted to the first filter media frame 128 through hinges 138 and 140. Preferably, as in the illustrated embodiment, the hinges 138, 140 pivot along a different axis than the access door hinge 188, for example along an axis that is perpendicular to that of the access door hinge 188 (e.g. whereby the access door 184 can swing open and closed in a substantially vertical movement and the second filter media frame 130 can swing open and closed in a substantially horizontal movement). The UV lamp 162 (a single lamp in this embodiment) is mounted by clips 166 to the back wall of the housing opposite the electrostatic filtration apparatus 111 and positioned to irradiate and disinfect air passing through the housing and optionally also disinfect the fibrous filter media 112 as discussed herein. A ballast 161 for lamp 162 is also mounted to an interior wall surface of housing 124.

In a vehicle installation of apparatus 180, as shown in FIGS. 13 and 14, the electrostatic filtration apparatus 111 and the UV source(s) can be electrically powered by the electrical system of the vehicle as discussed herein. The motors 183 for the fan(s) 182 can also be powered by the electrical system of the vehicle, for example by wiring an electrical power supply device for the apparatus 180 into the existing electrical system of the vehicle in a retrofit installation and powering the motor(s), electrostatic filtration apparatus 111 and the UV source(s) 162,164 through the power supply device.

With reference now to FIG. 15, illustrated is an embodiment of a railcar 200 (train car) having a roof-mounted heating, ventilation and air-conditioning (HVAC) system. Railcar has installed therein an air filtration and disinfection apparatus 160A which can be similar to apparatus 160 as described hereinabove, potentially wherein housing 124, grille 122 and padding 152 of apparatus 160 have been replaced by a mountable frame to which the other described components are attached. Apparatus 160A can be powered by the electrical system of the railcar 200, which can for example be a 24V DC electrical system as is typical in many railcars. In the illustrated embodiment, apparatus 160A is mounted above passenger compartment 202 in air return duct 204 and behind air return grille 206. The railcar HVAC system has a mixing chamber 208 where return air from passenger compartment 202 mixes with fresh air drawn from outside the railcar 200, and the mixed air passes through air filters 210 and 212 (which can have a MERV rating for example of 6 to 8) and thereafter to roof-mounted evaporator(s) and/or roof-mounted heater(s) prior to supply into passenger compartment 202 through supply grille(s) 214. UV source(s) 162, 164 can be positioned and configured to disinfect the return air from passenger compartment 202 flowing past them and in some embodiments can also be positioned and configured to disinfect the mixed return/fresh air in mixing chamber 208 or at least a portion thereof. In other embodiments herein, the railcar 200 or another vehicle similarly configured with an HVAC unit can be equipped with the electrostatic filter apparatus 111 of apparatus 160A but can lack the UV source(s) 162, 164. The electrostatic filter apparatus 111 (with or without the UV disinfection source) can serve to purify the air in the passenger compartment as well as protect components of the HVAC system against fouling with particulates.

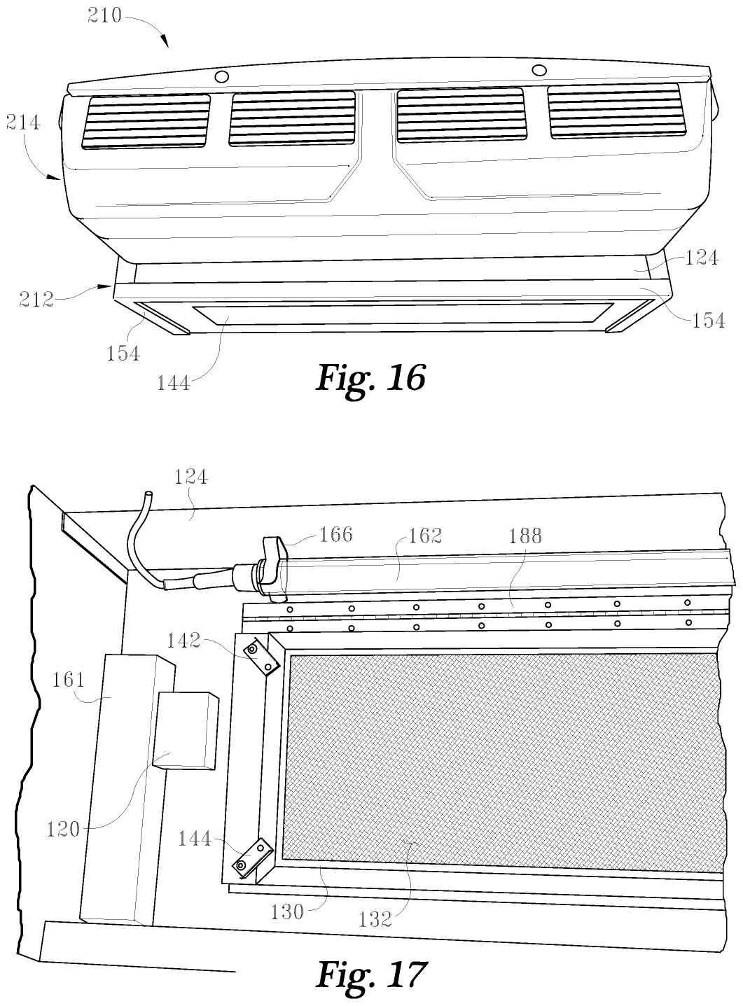

Referring now to FIGS. 16 and 17, shown is another apparatus for filtering and disinfecting air on a vehicle, for example a passenger bus. An overall air conditioning and purification system 210 includes a ceiling-mounted air conditioning unit 214 equipped with an air filtration and disinfection apparatus 212, such as described herein, to provide return air to the unit 214 (for example with apparatus 212 retrofitted over a return air opening of unit 214). Apparatus 212 includes UV source and filtration components similar to those of other embodiments herein, which are similarly numbered in FIGS. 16 and 17. Air conditioning unit 214 is similarly powered by the electrical system of the vehicle, and has supply air opening(s) 216 (e.g. covered with grille(s) or louver(s)) for supplying conditioned air to the passenger compartment of the vehicle. Air conditioning unit 214 houses therein fan(s) and associated motor(s) for powering air flow through unit 214, and can also house an evaporator coil in which refrigerant is evaporated to provide cooling to such air and/or a heater for heating such air. In some forms, a condenser for condensing the refrigerant can be fluidly connected to the evaporator and mounted on the roof of the vehicle. Other conventional HVAC components such as pumps and controls will of course also be present. Suitable ceiling-mounted air conditioning units such as unit 214 are available from commercial sources such as Thermo King of Minneapolis, Minn., USA, and can be fitted air filtration and/or disinfection apparatuses as disclosed herein.

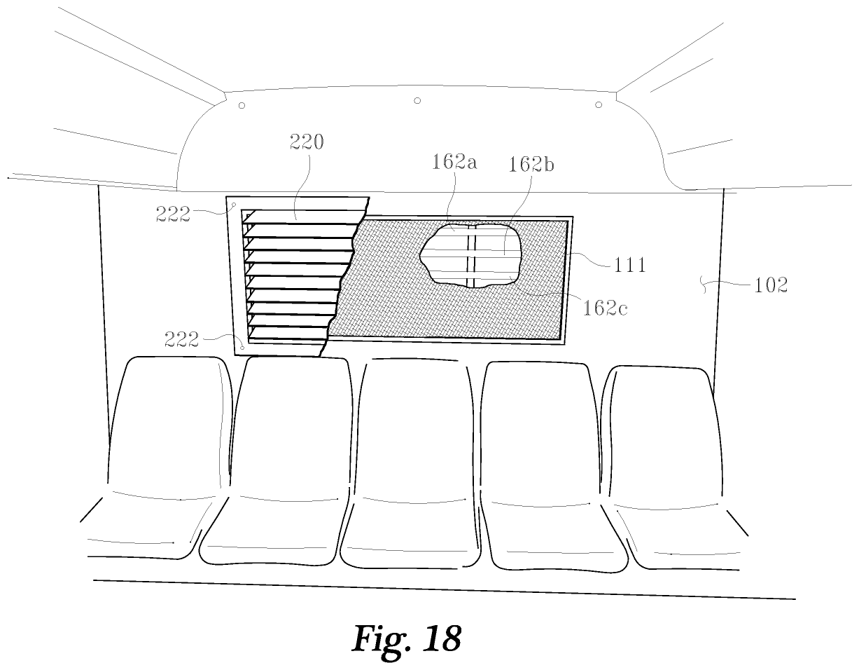

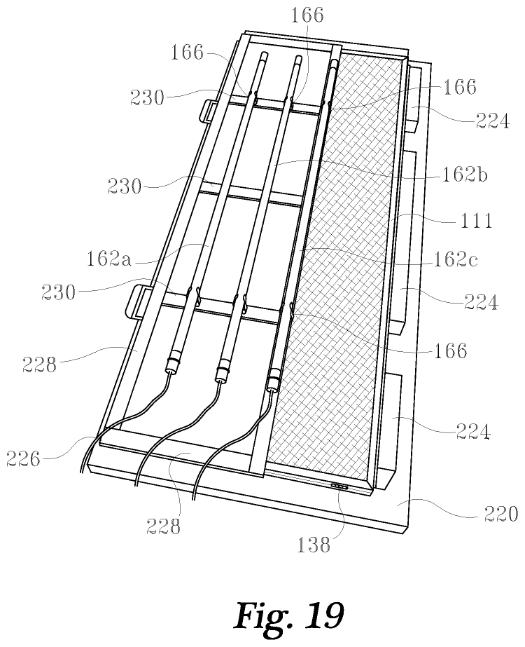

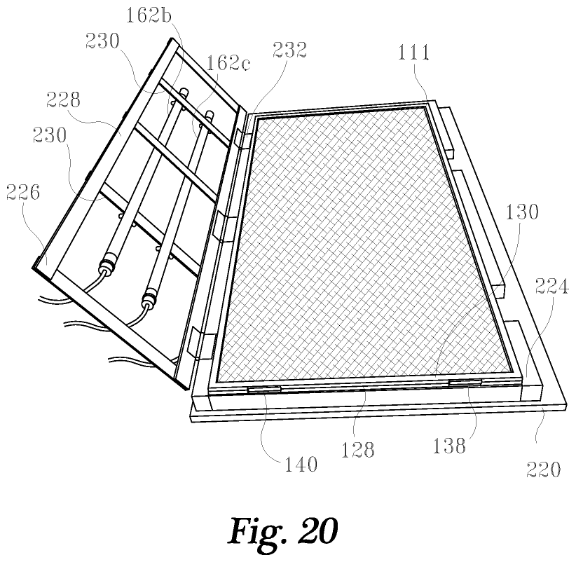

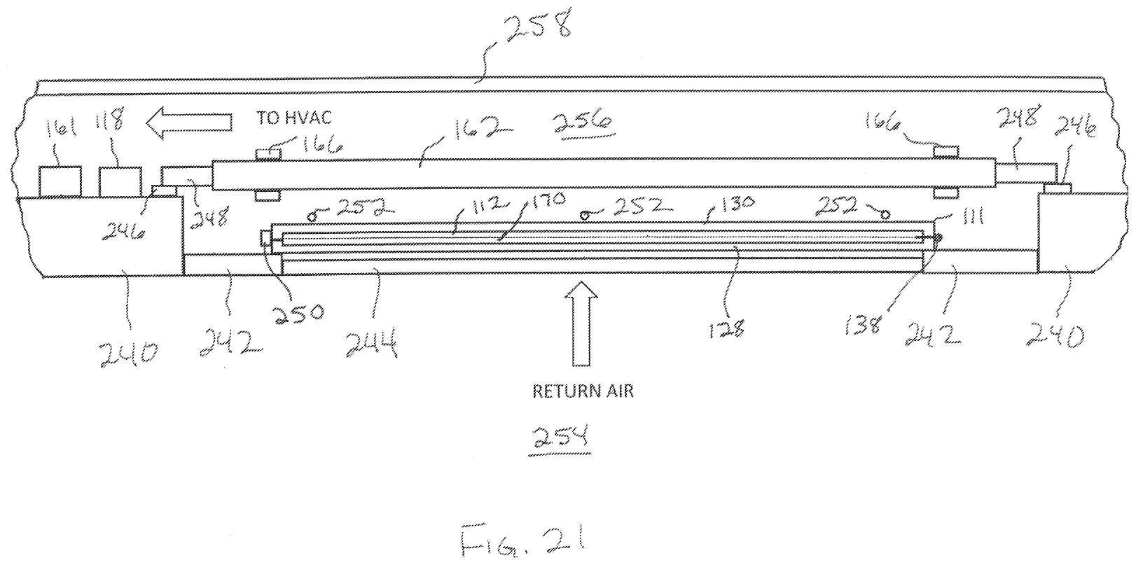

Referring now to FIGS. 18 to 20, shown is another embodiment of an apparatus for filtering and disinfecting air on a vehicle, for example a passenger bus. In the illustrated embodiment, a bus such as a metropolitan bus has a rear wall 102 having mounted thereto a return air grille 220 such as a polymeric or metal grille. For example, grille 220 can be mounted to a rear wall of the passenger compartment with screws 222 or other connectors, and an evaporator of an air conditioning system of the vehicle can be located behind the rear wall and can receive thereover air drawn through the grille 220 and treated by filtration and/or disinfection as described herein. FIG. 18 shows a partial cutaway view in two layers to show, respectively, components of the electrostatic filtration apparatus 111 and of the ultraviolet light disinfection unit including bulbs 162a, 162b, and 162c that are positioned behind the grille 220. As shown more clearly in FIGS. 19 and 20, a mounting frame, such as an aluminum frame 224, is mounted to the rear surface of the grille 220. The first frame member 128 of an electrostatic filtration apparatus 111 as described herein is attached to the mounting frame 224, and the second frame member 130 is hingedly attached to the first frame member as also described herein. A fibrous dielectric filter media 112 is received between the frame members 128 and 130, preferably along with one or more carbon fiber or other UV-blocking layers 170, more preferably wherein the layer(s) 170 are situated to the passenger compartment facing side of the filter media 112, as described above (see e.g. FIG. 12). Ultraviolet lamps 162a, 162b and 162c are mounted by clips 166 to bulb rack 226. Bulb rack 226 includes a peripheral frame 228 and crossbars 230 to which clips 166 are mounted. Bulb rack 226 is hingedly mounted to the frame member 130 by hinge 232, or alternatively can be hingedly mounted to the mounting frame 222 separate from the frame member 130. Bulb rack 226 can be removably held in its operating position (adjacent the electrostatic filter apparatus 111), for example by screws 228 or other connectors, which can be removed to allow rack 226 to swing or pivot away from electrostatic filter apparatus 111. In regard to the electrostatic filtration apparatus 111, as discussed above, frame member 130 can be swung or pivoted away from frame member 128 on hinges 138 and 140 (after release of stops 142 and 144), e.g. to replace filter media 112 and/or UV blocking fibrous layer(s) 170. Frame member 130 can pivot on a different axis than bulb rack 226, and in a preferred embodiment bulb rack 226 will swing in a vertical direction whereas frame member 130 will swing in a horizontal direction. The embodiment of FIGS. 18 to 20 can be powered by the electrical system of the vehicle as discussed herein (optionally with ballast(s) for ultraviolet lamp(s) mounted adjacent the grille 220, e.g. in a separate housing), and it will be understood that this embodiment can be otherwise configured as and perform as discussed for other air filtration and/or disinfection embodiments disclosed herein. The treated air can pass through the HVAC system of the vehicle for heating and/or cooling, and then be supplied into the passenger compartment through one or more appropriately located supply openings. To service the filtration and/or disinfection components of the apparatus, the grille 220 can be uninstalled from the rear wall 102, and the grille 220 and its mounted components removed for servicing and/or component replacements as discussed herein.

Referring now to FIG. 21, shown is a schematic representation of a side view of another embodiment of an apparatus for filtering and disinfecting air on a vehicle, for example a passenger bus. In the illustrated embodiment, a bus such as a metropolitan bus has a ceiling wall 240 having hingedly mounted therein an access door that includes a peripheral frame 242 defining a return air opening covered by a grille 244 for passage of return air (which can for example be a mesh material). A bulb rack for holding ultraviolet bulb(s) 162 is mounted to an upper surface of ceiling wall 240 and includes a peripheral frame 246 and crossbar(s) 248 spanning the peripheral frame 246. While in the depicted side view only a single crossbar 248 and bulb 162 is visible, in preferred forms a plurality of crossbars 248 and respective supported bulbs 162 are provided (e.g. 2, 3 or 4 ultraviolet bulbs spanning the width of grille 244). Bulb mounting clips 166 are mounted to the crossbar(s) 248 (preferably with their clip openings directed horizontal as shown for horizontally directed receipt of ultraviolet bulb(s) 162. A ballast(s) 161 for energizing lamp(s) can be mounted to an upper surface of ceiling wall 240 and can be connected to bulb(s) 162 with an electrical wire(s) as is conventional. The installed apparatus also includes an electrostatic filtration unit 111 generally as described previously herein. Apparatus 111 includes a fibrous dielectric filter media 112 and optionally also UV-blocking fibrous material 170 as described herein (see e.g. FIG. 12 or 12A). The filter media 112, a distributor element 114 (see prior discussions and figures), and when present UV-blocking fibrous material 170 are supported between frame members 128 and 130, which are hingedly connected for example by hinge 138 and hinge 140 (latter not shown in FIG. 21) so that frame members 128 and 130 can be separated to access and replace the filter media 112 and/or UV-blocking fibrous material 170. A latch 250 can be provided at the end of frames 128 and 130 opposite the hinge(s) 138, 140 and can be operable to reversibly secure them together. A power supply 118 for the electrostatic filter apparatus 111 can be mounted to an upper surface of the ceiling wall 240. It will be understood that power supply 118 will be wired to a contact probe 116 providing voltage to the distributor element 114 as discussed hereinabove. In the illustrated embodiment the electrostatic filter apparatus is secured by a strap connected to the ceiling-mounted access door (e.g. to peripheral frame 242 thereof), with strap 250 being repeatedly strung across an upper surface of apparatus 111. In operation, return air from passenger compartment 254 passes through grille 244 and into space 256 occurring between ceiling wall 240 and roof 258 of the vehicle (bus), where it is filtered and disinfected as described herein. The air then passes to components of the HVAC system for cooling (e.g. an evaporator) or heating (e.g. a heater), these components for example occurring at a more forward position in the vehicle relative to the electrostatic air filtration and disinfection components shown. The treated air can then be supplied into the passenger compartment through one or more appropriately located supply openings. The embodiment of FIG. 21 can be powered by the electrical system of the vehicle as discussed herein, and it will be understood that this embodiment can be otherwise configured as and perform as discussed for other air filtration and/or disinfection embodiments disclosed herein. To service the filtration and/or disinfection components of the apparatus, the access door including frame 242 and grille 244 can be hingedly opened, with the electrostatic filtration apparatus 111 travelling with the access door. Strap 252 can be removed to provide access to unlatch and separate frame elements 128 and 130 to change the media therein. The bulb(s) will remain on their rack mounted to the ceiling wall 240 and can be replaced or otherwise serviced with the access door open.

In embodiments disclosed herein including one or more ultraviolet lamps, any ballast(s) for the lamps can be mounted along with the lamps (e.g. mounted to a frame to which the lamps are mounted as discussed herein), or can be separately mounted either within any disclosed housing in which the lamps are situated or outside such housing (e.g. in a separate housing of their own). As will be understood, appropriate wiring can be provided to electrically connect the ballasts to an electrical power source such as any described herein. As well, it will be understood that in some variations ultraviolet lamp(s) can be unplugged from respective ballast(s) for servicing or replacement operations as described herein, for example at the ballast side or at the lamp side of an electrical wire(s) connecting the lamp(s) and ballast(s).

When installed in a vehicle to treat the air circulating through a passenger compartment, the embodiments herein can be installed at any suitable location for doing so. For example, installation in a wall that provides a sidewall, ceiling and/or floor of the passenger compartment is contemplated.

In still further embodiments herein, in addition to or instead or utilizing ultraviolet light source(s) to inactivate viral particles and/or other pathogens, one or more bipolar ionization elements can be used. Bipolar ionization elements have high voltage wires or other members that generate air that is rich in positive and negative oxygen ions. The negative ions contain an extra electron while the positive ions are missing an electron resulting in an unstable condition. In an effort to restabilize, these bipolar ions seek out sources of electron exchange, effectively neutralizing viruses and other potential pathogens in the air. For additional information regarding bipolar ionization elements that can be used, reference can be made for example to United States Patent Publication No. 2010247389A1 published Sep. 30, 2010 and United States Patent Application Publication No. 2012287551A1 published Nov. 15, 2012, each of which is hereby incorporated herein by reference in its entirety. In these embodiments, some or all of the elements 12 depicted in the Figures can represent bipolar ionization elements instead of ultraviolent light sources. Combinations of one or more ultraviolet light sources and one or more bipolar ionization elements can be used in some embodiments herein.

While embodiments of the disclosure have been illustrated and described in detail in the drawings and foregoing description, the same is to be considered as illustrative and not restrictive in character, it being understood that only some embodiments have been shown and described and that all changes and modifications that come within the spirit of the disclosures herein are desired to be protected. As examples, the following Listing of Embodiments provides an identification of some of the embodiments disclosed herein. It will be understood that this listing is non-limiting, and that individual features or combinations of features (e.g. 2, 3 or 4 features) as described in the Detailed Description above can be combined with the below-listed embodiments to provide additional disclosed embodiments herein.

### ENUMERATED LISTING OF CERTAIN DISCLOSED EMBODIMENTS

1. A vehicle having an onboard air purification system that inactivates virus particles, comprising:

a vehicle having an air conditioned interior passenger compartment, the vehicle having a first air intake opening in a wall through which air is removed from an interior passenger compartment of the vehicle for conditioning by a heating and/or cooling system, and a first air outflow opening through which the air is returned to the interior after conditioning by the heating and/or cooling system;

a housing mounted to the wall and including an air intake grille communicating with the interior passenger compartment and an air passage opening communicating with the first air intake opening, the housing being arranged so that the air removed from the interior passenger compartment passes through the housing and then through the air intake opening;

an ultraviolet light source and/or a bipolar ionization element mounted within the housing and effective to inactivate virus particles in air passing through the housing in a path from the air intake grille of the housing to the air passage opening of the housing.

2. The vehicle of embodiment 1, wherein the vehicle is a bus, preferably a school bus.

3. The vehicle of embodiment 1 or 2, wherein the wall is a back wall or front wall bounding the interior passenger compartment.

4. The vehicle of any preceding embodiment, including the ultraviolet light source mounted within the housing.

5. The vehicle of embodiment 4, wherein the ultraviolet light source emits ultraviolet light with a wavelength of in the range between 200-280 nanometers.

6. The vehicle of embodiment 4 or 5, wherein the ultraviolet light source has a peak emission at about 254 nanometer and/or irradiates ultraviolet light at an intensity of at least about 200 microwatts/cm2, or in the range of about 200 to about 300 microwatts/cm².

7. The vehicle of embodiment 4, 5 or 6, wherein the ultraviolet light source is an ultraviolet lamp.

8. The vehicle of any preceding embodiment, wherein the housing has an interior volume capacity of at least about 500 cubic inches, or in the range of about 500 to about 3000 cubic inches, or in the range of about 1000 to about 1700 cubic inches.

9. The vehicle of any preceding embodiment, wherein the heating and/or cooling system is configured to change the air of the interior passenger compartment at a rate of at least about 50 air changes per hour, or in the range of about 50 to about 150 changes per hour, or in the range of about 100 to 150 air changes per hour.

10. The vehicle of any preceding embodiment, wherein the housing has a hingedly connected panel for providing access to the interior of the housing.

11. The vehicle of embodiment 10, also comprising a switch electrically connected to an electrical power source for the ultraviolet light or the bipolar ionization element, the switch arranged to interrupt power to the ultraviolet light or the bipolar ionization element when the panel is opened.

12. The vehicle of any preceding embodiment, wherein the heating and/or cooling system and the housing are sized and configured to pass air through the housing at a rate of at least 800 feet per minute, preferably in the range of about 800 to about 1500 feet per minute, and more preferably about 1000 to about 1500 feet per minute.

13. The vehicle of any preceding embodiment, also including a washable filter mounted in the housing through which the air passes after passage through the air intake grille and before passage by the ultraviolet light source or bipolar ionization element.

14. The vehicle of any preceding embodiment, which is equipped to generate direct electrical current, wherein the ultraviolet light or the bipolar ionization element operates on alternating current, and wherein the vehicle also includes a DC to AC inverter for converting the generated direct current to alternating current for powering the ultraviolet light or the bipolar ionization element.

15. The vehicle of any preceding embodiment, wherein the air intake grill prevents direct passage of UV rays from within the housing.

16. The vehicle of embodiment 15, wherein the air intake grill includes a plurality of grill elements each having a first portion extending in a first direction, a second portion extending in a second direction at an angle relative to the first direction, and a peak connecting the first portion and the second portion.

17. The vehicle of any preceding embodiment, wherein the air intake grille is removably mounted to the housing.

18. The vehicle of any preceding embodiment, also including a washable filter mounted to the housing.

19. The vehicle of any preceding embodiment, wherein the first air intake opening and the first air outflow opening are in a back wall of a passenger compartment of the vehicle.

20. A method for retrofitting a vehicle to provide a retrofitted vehicle capable of viral inactivation, the vehicle having an air conditioned interior passenger compartment, the vehicle having a first air intake opening in a wall through which air is removed from an interior passenger compartment of the vehicle for conditioning by a heating and/or cooling system, and a first air outflow opening through which the air is returned to the interior after conditioning by the heating and/or cooling system, the method comprising;

mounting a housing to the wall, the housing including an air intake grille for communicating with the interior passenger compartment and an air passage opening for communicating with the first air intake opening, the housing being arranged and mounted to the wall so that the air removed from the interior passenger compartment passes through the housing and then through the air intake opening; and

providing an ultraviolet light source and/or a bipolar ionization element mounted within the housing and effective to inactivate virus particles in air passing through the housing in a path from the air intake grille of the housing to the air passage opening of the housing.

21. The method of embodiment 20, wherein the retrofitted vehicle is a vehicle according to any one of embodiments 2 to 19.

22. A method for inactivating virus particles in air in an interior passenger compartment of a vehicle, the vehicle having a first air intake opening in a wall through which air is removed from the interior passenger compartment of the vehicle for conditioning by a heating and/or cooling system of the vehicle, and a first air outflow opening through which the air is returned to the interior passenger compartment after conditioning by the heating and/or cooling system, the method comprising;

passing the air which is removed from the interior passenger compartment through a housing mounted to the wall and including an air intake grille communicating with the interior passenger compartment and an air passage opening communicating with the first air intake opening, the housing being arranged so that the air removed from the interior passenger compartment passes through the housing and then through the air intake opening; and

treating the air passing through the housing with an ultraviolet light source and/or a bipolar ionization element mounted within the housing so as to inactivate virus particles.

23. The method of embodiment 22, wherein the vehicle is a bus, preferably a school bus.

24. The method of embodiment 22 or 23, wherein the wall is a back wall or front wall bounding the interior passenger compartment.

25. The method of any one of embodiments 22 to 24, wherein the treating includes treating with the ultraviolet light source.

26. The method of embodiment 25, wherein the ultraviolet light source emits ultraviolet light with a wavelength of in the range between 200-280 nanometers.

27. The method of embodiment 25 or 26, wherein the ultraviolet light source has a peak emission at about 254 nanometer and/or irradiates ultraviolet light at an intensity of at least about 200 microwatts/cm², or in the range of about 200 to about 300 microwatts/cm².

28. The vehicle of embodiment 4, 5 or 6, wherein the ultraviolet light source is an ultraviolet lamp.

29. The method of any one of embodiments 22 to 28, wherein the housing has an interior volume capacity of at least about 500 cubic inches, or in the range of about 500 to about 3000 cubic inches, or in the range of about 1000 to about 1700 cubic inches.

30. The method of any one of embodiments 22 to 29, wherein the heating and/or cooling system is configured to change the air of the interior passenger compartment at a rate of at least about 50 air changes per hour, or in the range of about 50 to about 150 changes per hour, or in the range of about 100 to 150 air changes per hour.

31. The method of any one of embodiments 22 to 30, wherein the housing has a hingedly connected panel for providing access to the interior of the housing.

32. The method of embodiment 31, wherein there is a switch electrically connected to an electrical power source for the ultraviolet light or the bipolar ionization element, the switch arranged to interrupt power to the ultraviolet light or the bipolar ionization element when the panel is opened.

33. The method of any one of embodiments 22 to 32, wherein said passing the air includes passing the air through the housing at a rate of at least 800 feet per minute, preferably in the range of about 800 to about 1500 feet per minute, and more preferably about 1000 to about 1500 feet per minute.

34. The method of any one of embodiments 22 to 33, also including providing a washable filter mounted in the housing through which the air passes after passage through the air intake grille and before passage by the ultraviolet light source or bipolar ionization element.

35. The method of any one of embodiments 22 to 34, wherein the vehicle is equipped to generate direct electrical current, wherein the ultraviolet light or the bipolar ionization element operates on alternating current, and wherein the vehicle also includes a DC to AC inverter for converting the generated direct current to alternating current for powering the ultraviolet light or the bipolar ionization element.

36. A kit for retrofitting a vehicle for viral inactivation, the vehicle having an air conditioned interior passenger compartment, the vehicle having a first air intake opening in a wall through which air is removed from the interior passenger compartment for conditioning by a heating and/or cooling system, and a first air outflow opening through which the air is returned to the interior passenger compartment after conditioning by the heating and/or cooling system, the kit comprising:

a housing mountable to the wall and including an air intake grille for communicating with the interior passenger compartment and an air passage opening for communicating with the first air intake opening, the housing being arranged so that when mounted to the wall the air removed from the interior passenger compartment passes through the housing and then through the air intake opening; and

an ultraviolet light source and/or a bipolar ionization element mounted or mountable within the housing and effective to inactivate virus particles in air passing through the housing in a path from the air intake grille of the housing to the air passage opening of the housing.

37. The kit of embodiment 36, wherein the vehicle is a bus, preferably a school bus.

38. The kit of embodiment 36 or 37, wherein the wall is a back wall or front wall bounding the interior passenger compartment.

39. The kit of any one of embodiments 36 to 38, including the ultraviolet light source mounted within the housing.

40. The kit of embodiment 39, wherein the ultraviolet light source emits ultraviolet light with a wavelength of in the range between 200-280 nanometers.

41. The kit of embodiment 39 or 40, wherein the ultraviolet light source has a peak emission at about 254 nanometer and/or irradiates ultraviolet light at an intensity of at least about 200 microwatts/cm2, or in the range of about 200 to about 300 microwatts/cm².

42. The kit of embodiment 39, 40 or 41, wherein the ultraviolet light source is an ultraviolet lamp.

43. The kit of any one of embodiments 36 to 42, wherein the housing has an interior volume capacity of at least about 500 cubic inches, or in the range of about 500 to about 3000 cubic inches, or in the range of about 1000 to about 1700 cubic inches.

44. The kit of any one of embodiments 36 to 43, wherein the heating and/or cooling system is configured to change the air of the interior passenger compartment at a rate of at least about 50 air changes per hour, or in the range of about 50 to about 150 changes per hour, or in the range of about 100 to 150 air changes per hour.

45. The kit of any one of embodiments 36 to 44, wherein the housing has a hingedly connected panel for providing access to the interior of the housing.

46. The kit of embodiment 45, also comprising a switch arranged to interrupt power to the ultraviolet light or the bipolar ionization element when the panel is opened.

47. The kit of any one of embodiments 36 to 46, wherein the heating and/or cooling system and the housing are sized and configured to pass air through the housing at a rate of at least 800 feet per minute, preferably in the range of about 800 to about 1500 feet per minute, and more preferably about 1000 to about 1500 feet per minute.

48. The kit of any one of embodiments 36 to 47, also including a washable filter mounted in the housing through which the air passes after passage through the air intake grille and before passage by the ultraviolet light source or bipolar ionization element.

49. The kit of any one of embodiments 36 to 48, wherein the vehicle is equipped to generate direct electrical current, wherein the ultraviolet light or the bipolar ionization element operates on alternating current, and wherein the kit also includes a DC to AC inverter for converting the generated direct current to alternating current for powering the ultraviolet light or the bipolar ionization element.

50. A vehicle having an onboard air treatment system that inactivates virus particles, comprising:

a vehicle having a passenger compartment, the vehicle having an air return opening through which air is removed from an interior passenger compartment of the vehicle for travel through an air circulation system, and an air supply opening through which the air is supplied to the interior after travel through the air circulation system; and

an ultraviolet light source and/or a bipolar ionization element positioned and effective to inactivate virus particles in the air.

51. The vehicle of embodiment 50, comprising said ultraviolet light source, and also comprising:

a return grille over the return opening; and

means for blocking ultraviolet radiation positioned between the return grille and the ultraviolet light source, said means for blocking optionally mounted to the return grille.

52. The vehicle of embodiment 51, wherein the return grille has a return grille outer surface, and wherein the intensity of ultraviolet light at a wavelength of 254 nanometers emitted by the ultraviolet light source that is measurable at the return grille outer surface is less than about 5 μW/cm², or less than about 3 μW/cm², or less than about 2 μW/cm², or less than about 1 μW/cm².

53. The vehicle of embodiment 52, wherein said intensity is less than about 0.5 μW/cm², or less than about 0.1 μW/cm², or less than about 0.01 μW/cm², or is zero.

54. The vehicle of any one of embodiments 50 to 53, wherein said ultraviolet light source has a peak emission at about 254 nanometer and/or irradiates ultraviolet light at an intensity of at least about 200 μW/cm², or in the range of about 200 to about 300 μW/cm².

55. The vehicle of any one of embodiments 50 to 54, also comprising an electrostatic filter element including a fibrous dielectric filter media to which voltage is applied to polarize the fibrous dielectric filter media, the electrostatic filter element positioned so that the air passes through the fibrous dielectric filter media for filtration.

56. The vehicle of embodiment 55, wherein the electrostatic filter element has a MERV equivalent rating of MERV-13 equivalent or greater.

57. The vehicle of embodiment 55 or 56, wherein said voltage is in the range of 5000 to 8000 volts.

58. The vehicle of any one of embodiments 55 to 57, wherein the air passes through the electrostatic filter element before passing by the ultraviolet light source and/or bipolar ionization element.

59. The vehicle of any one of embodiments 55 to 58, comprising the ultraviolet light source, and also comprising a secondary fibrous media associated with the fibrous dielectric filter media, the secondary fibrous media being less penetrable by the ultraviolet light than the fibrous dielectric filter media.

60. The vehicle of embodiment 59, wherein the secondary fibrous media is an electrically non-conductive fibrous media.

61. The vehicle of embodiment 59 or 60, wherein the secondary fibrous media is comprised of carbon fibers, and preferably wherein the fibrous dielectric filter media is a fiberglass filter media.

62. The vehicle of any one of embodiments 59 to 60, wherein the secondary fibrous media is positioned adjacent the fibrous dielectric filter media to an opposite side of the fibrous dielectric filter media relative to the ultraviolet light source.

63. The vehicle of any one of embodiments 59 to 61, comprising a first frame member and a second frame member, with said fibrous dielectric filter media and said secondary fibrous media positioned between the first frame member and second frame member.

64. The vehicle of embodiment 63, wherein the ultraviolet light source is mounted to the second frame member.

65. The vehicle of embodiment 63 or 64, wherein the first frame member is hingedly mounted to the second frame member, preferably wherein the ultraviolet light source comprises a plurality of ultraviolet lamps.

66. The vehicle of any one of embodiments 59 to 65, wherein said ultraviolet light source is effective to irradiate and inactivate virus particles on the fibrous dielectric filter media.

67. An apparatus for installation in a vehicle to treat air of a passenger compartment of the vehicle, comprising:

an ultraviolet light source and/or a bipolar ionization element positionable to inactivate virus particles in the air; and

a mount member mountable to the vehicle and supporting the ultraviolet light source and/or bipolar ionization element.

68. The apparatus of embodiment 67, comprising said ultraviolet light source, and also comprising:

a return grille for mounting over a return air opening into the passenger compartment; and

means for blocking ultraviolet radiation positionable between the return grille and the ultraviolet light source, said means for blocking optionally mounted to the return grille.

69. The apparatus of embodiment 69, wherein the mount member is a housing including the return grille and having the ultraviolet light source mounted herein, wherein the return grille has a return grille outer surface, wherein the ultraviolet light source is configured when energized to emit ultraviolet light including a wavelength of 254 nanometers, and wherein an intensity of the emitted ultraviolet light at a wavelength of 254 nanometers that is measurable at the return grille outer surface is less than about 5 μW/cm², or less than about 3 μW/cm², or less than about 2 μW/cm², or less than about 1 μW/cm².

70. The apparatus of embodiment 69, wherein said intensity is less than about 0.5 μW/cm², or less than about 0.1 μW/cm², or less than about 0.01 μW/cm², or is zero.

71. The apparatus of any one of embodiments 67 to 70, wherein said ultraviolet light source has a peak emission at about 254 nanometer and/or irradiates ultraviolet light at an intensity of at least about 200 μW/cm², or in the range of about 200 to about 300 μW/cm².

72. The apparatus of any one of embodiments 67 to 71, also comprising an electrostatic filter element including a fibrous dielectric filter media to which voltage is applied to polarize the fibrous dielectric filter media, the electrostatic filter element positioned so that the air passes through the fibrous dielectric filter media for filtration.

73. The apparatus of embodiment 72, wherein the electrostatic filter element has a MERV equivalent rating of MERV-13 equivalent or greater.

74. The apparatus of embodiment 72 or 73, wherein said voltage is in the range of 5000 to 8000 volts.

75. The apparatus of any one of embodiments 72 to 74, wherein the air passes through the electrostatic filter element before passing by the ultraviolet light source and/or bipolar ionization element.

76. The apparatus of any one of embodiments 72 to 75, comprising the ultraviolet light source, and also comprising a secondary fibrous media associated with the fibrous dielectric filter media, the secondary fibrous media being less penetrable by the ultraviolet light than the fibrous dielectric filter media.

77. The apparatus of embodiment 76, wherein the secondary fibrous media is an electrically non-conductive fibrous media.

78. The apparatus of embodiment 76 or 77, wherein the secondary fibrous media is comprised of carbon fibers, and preferably wherein the fibrous dielectric filter media is a fiberglass filter media.

79. The apparatus of any one of embodiments 76 to 78, wherein the secondary fibrous media is positioned adjacent the fibrous dielectric filter media to an opposite side of the fibrous dielectric filter media relative to the ultraviolet light source.

80. The apparatus of any one of embodiments 76 to 79, comprising a first frame member and a second frame member, with said fibrous dielectric filter media and said secondary fibrous media positioned between the first frame member and second frame member.

81. The apparatus of embodiment 80, wherein the ultraviolet light source is mounted to the second frame member.

82. The apparatus of embodiment 80 or 81, wherein the first frame member is hingedly mounted to the second frame member, preferably wherein the ultraviolet light source comprises a plurality of ultraviolet lamps.

83. The apparatus of any one of embodiments 72 to 82, wherein said ultraviolet light source is effective to irradiate and inactivate virus particles on the fibrous dielectric filter media.

84. The apparatus of any one of embodiments 67 to 83, also comprising at least one electric fan mounted to the mount member.

85. The apparatus of embodiment 84, wherein the mount member is a housing, wherein the housing has an air return opening for receiving air from the passenger compartment and an air supply opening for supplying air to the passenger compartment, and wherein the at least one electric fan is effective to power air flow within the housing in a path from the air return opening to and out of the air supply opening.

86. The apparatus of any one of embodiments 72 to 85, wherein the mount member is a housing having a housing access door for accessing an interior of the housing.

87. The apparatus of embodiment 86, wherein the electrostatic filter element is mounted to the housing access door.

88. The apparatus of embodiment 86 or 87, wherein the ultraviolet light source and/or bipolar ionization element is mounted to the housing access door.

89. The apparatus of embodiment 87 or 88, wherein the electrostatic filter element is retained by a filter frame mounted to the housing access door, and wherein the ultraviolet light source and/or bipolar ionization element is mounted to the filter frame.

90. The apparatus of embodiment 89, wherein the filter frame includes a first filter frame member fixedly mounted to the access door and a second filter frame member hingedly mounted to the first filter frame member, and wherein the ultraviolet light source and/or bipolar ionization element is mounted to and travels with the second filter frame member.

91. The apparatus of embodiment 90, wherein the housing access door is hingedly mounted to the housing.

92. The apparatus of embodiment 91, wherein the housing access door is arranged to pivot on a first axis and the second filter frame member is arranged to pivot on a second axis, wherein the first axis differs from the second axis.

93. An apparatus for installation in a vehicle to treat air circulated through a passenger compartment of the vehicle, comprising:

an electrostatic filter element including a dielectric filter media to which voltage is applied to polarize the dielectric filter media, the electrostatic filter element positionable so that the air passes through the dielectric filter media for filtration; and

a frame mountable to the vehicle and supporting the electrostatic filter element. 94. The apparatus of embodiment 93, wherein the electrostatic filter element has a MERV equivalent rating of MERV-13 equivalent or greater.

95. The apparatus of embodiment 93 or 94, wherein said voltage is in the range of 5000 to 8000 volts.

96. The apparatus of any one of embodiments 93 to 95, comprising a first frame member and a second frame member, with said fibrous dielectric filter media positioned between the first frame member and second frame member.

97. The apparatus of embodiment 96, also comprising an ultraviolet light source is mounted to the second frame member, the ultraviolet light source effective to inactivate viral particles.

98. The apparatus of embodiment 96 or 97, wherein the first frame member is hingedly mounted to the second frame member.

99. The apparatus of any one of embodiments 93 to 98, also comprising at least one electric fan mounted to the housing.

100. The apparatus of embodiment 99, wherein the housing has an air return opening for receiving air from the passenger compartment and an air supply opening for supplying air to the passenger compartment, and wherein the at least one electric fan is effective to power air flow within the housing in a path from the air return opening, through the fibrous dielectric filter media, and out of the air supply opening.

101. The apparatus of any one of embodiments 93 to 100, wherein the housing has a housing access door for accessing an interior of the housing.

102. The apparatus of embodiment 101, wherein the electrostatic filter element is mounted to the housing access door.

103. The apparatus of embodiment 102, wherein the electrostatic filter element is retained by a filter frame mounted to the housing access door.

104. The apparatus of embodiment 103, wherein the filter frame includes a first filter frame member fixedly mounted to the access door and a second filter frame member hingedly mounted to the first filter frame member.

105. The apparatus of embodiment 104, wherein the housing access door is hingedly mounted to the housing.

106. The apparatus of embodiment 105, wherein the housing access door is arranged to pivot on a first axis and the second filter frame element is arranged to pivot on a second axis, wherein the first axis differs from the second axis.

107. An apparatus for treating air, comprising:

an electrostatic filter element including a fibrous dielectric filter media to which voltage is applied to polarize the dielectric filter media, the electrostatic filter element positionable so that the air passes through the fibrous dielectric filter media for filtration, the air entering the fibrous dielectric filter media at a first surface thereof and exiting the fibrous dielectric filter media at a second surface thereof;

a secondary fibrous media that is less penetrable to the ultraviolet light than the fibrous dielectric filter media; and

an ultraviolet light source that emits ultraviolet light, the ultraviolet light source positioned and effective to inactivate virus particles in the air and to irradiate the fibrous dielectric filter media with the ultraviolet light in a direction from a the second side of the fibrous dielectric filter media to the first side of the fibrous dielectric filter media; and

wherein the secondary fibrous media is effective to reduce the intensity of the ultraviolet light at a position spaced outwardly from the first surface of the fibrous dielectric media.

108. The apparatus of embodiment 107, wherein the electrostatic filter element has a MERV equivalent rating of MERV-13 equivalent or greater.

109. The apparatus of embodiment 107 or 108, wherein said voltage is in the range of 5000 to 8000 volts.

110. The apparatus of any one of embodiments 107 to 109, wherein the air passes through the electrostatic filter element before passing by the ultraviolet light source and/or bipolar ionization element.

111. The apparatus of any one of embodiments 107 to 110, also comprising a return grille through which air passes to be treated by the electrostatic filter element and the ultraviolet light source, the return grille having a return grille outer surface, and wherein the intensity of ultraviolet light at a wavelength of 254 nanometers emitted by the ultraviolet light source that is measurable at the return grille outer surface is less than about 5 μW/cm², or less than about 3 μW/cm², or less than about 2 μW/cm², or less than about 1 μW/cm².

112. The apparatus of embodiment 111, wherein said intensity is less than about 0.5 μW/cm², or less than about 0.1 μW/cm², or less than about 0.01 μW/cm².

113. The apparatus of embodiment 112, wherein said intensity is zero.

114. The apparatus of any one of embodiments 107 to 113, wherein said ultraviolet light source has a peak emission at about 254 nanometer and/or irradiates ultraviolet light at an intensity of at least about 200 μW/cm², or in the range of about 200 to about 300 μW/cm².

115. The apparatus of any one of embodiments 107 to 114, wherein the secondary fibrous media is an electrically non-conductive fibrous media.

116. The apparatus of any one of embodiments 107 to 115, wherein the secondary fibrous media is comprised of carbon fibers, and preferably wherein the fibrous dielectric filter media is a fiberglass filter media.

117. The apparatus of any one of embodiments 107 to 116, wherein the secondary fibrous media is in the form of a layer positioned adjacent the fibrous dielectric filter media to an opposite side of the fibrous dielectric filter media relative to the ultraviolet light source.

118. The apparatus of any one of embodiments 107 to 117, comprising a first frame member and a second frame member, with said fibrous dielectric filter media and said secondary fibrous media positioned between the first frame member and second frame member.

119. The apparatus of embodiment 118, wherein the ultraviolet light source is mounted to the second frame member.

120. The apparatus of embodiment 118 or 119, wherein the first frame member is hingedly mounted to the second frame member.

121. The apparatus of any one of embodiments 107 to 120, wherein the ultraviolet light source comprises a plurality of ultraviolet lamps.

122. The apparatus of any one of embodiments 107 to 121, wherein said ultraviolet light source is effective to irradiate and inactivate virus particles on the fibrous dielectric filter media.

123. The apparatus of any one of embodiments 107 to 122, also comprising a housing, with said electrostatic filter element and said ultraviolet light source within the housing.

124. The apparatus of embodiment 123, wherein the housing has an air return opening for receiving air to be treated and an air supply opening for supplying air after treatment.

125. The apparatus of embodiment 124, also comprising at least one electric fan effective to power air flow within the housing in a path from the air return opening to the air supply opening.

126. The apparatus of any one of embodiments 107 to 125, wherein the housing has a housing access door for accessing an interior of the housing.

127. The apparatus of embodiment 126, wherein the electrostatic filter element is mounted to the housing access door.

128. The apparatus of embodiment 126 or 127, wherein the ultraviolet light source is mounted to the housing access door.

129. The apparatus of embodiment 128, wherein the electrostatic filter element is retained by a filter frame mounted to the housing access door, and wherein the ultraviolet light source and/or bipolar ionization element is mounted to the filter frame.

130. The apparatus of embodiment 129, wherein the filter frame includes a first filter frame member fixedly mounted to the access door and a second filter frame member hingedly mounted to the first filter frame member, and wherein the ultraviolet light source and/or bipolar ionization element is mounted to and travels with the second filter frame member.

131. The apparatus of embodiment 130, wherein the housing access door is hingedly mounted to the housing.

132. The apparatus of embodiment 131, wherein the housing access door is arranged to pivot on a first axis and the second filter frame member is arranged to pivot on a second axis, wherein the first axis differs from the second axis.

133. A method for treating air of a passenger compartment of a vehicle, comprising:

passing the air through a return grille exposed to the passenger compartment;

after said passing, irradiating the air with ultraviolet light to inactivate viral particles in the air;

blocking the ultraviolet light from entering the passenger compartment with means for blocking ultraviolet light, the means for blocking ultraviolet light associated with the return grille; and

after said irradiating, supplying the air to the passenger compartment through a supply opening into the passenger compartment.

134. The method of embodiment 133, also comprising, after said passing and before said supplying, filtering the air.

135. The method of embodiment 134, wherein the filtering comprises flowing the air through an electrostatic filter element including a fibrous dielectric filter media to which a voltage is applied to polarize the filter media.

136. The method of embodiment 135, wherein the blocking comprises blocking the ultraviolet light with a secondary fibrous media associated with the fibrous dielectric filter media.

137. The method of any one of embodiments 133 to 136, wherein the return grille has a return grille outer surface, and wherein the intensity of ultraviolet light at a wavelength of 254 nanometers emitted by the ultraviolet light source that is measurable at the return grille outer surface is less than about 5 μW/cm2, or less than about 3 μW/cm2, or less than about 2 μW/cm2, or less than about 1 μW/cm2.

138. The method of embodiment 137, wherein said intensity is less than about 0.5 μW/cm2, or less than about 0.1 μW/cm2, or less than about 0.01 μW/cm2.

139. The method of embodiment 137 or 138, wherein said intensity is zero.

The uses of the terms “a” and “an” and “the” and similar references herein (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the disclosure and does not pose a limitation on the scope of the disclosure unless otherwise claimed. No language in the specification should be construed as indicating any non-claimed element as essential to the practice of the products or methods defined by the claims.

1. A vehicle having an onboard air purification system that inactivates virus particles, comprising:
a vehicle having an air conditioned interior passenger compartment, the vehicle having a first air intake opening in a wall through which air is removed from an interior passenger compartment of the vehicle for conditioning by a heating and/or cooling system, and a first air outflow opening through which the air is returned to the interior after conditioning by the heating and/or cooling system;
a housing mounted to the wall and including an air intake grille communicating with the interior passenger compartment and an air passage opening communicating with the first air intake opening, the housing being arranged so that the air removed from the interior passenger compartment passes through the housing and then through the air intake opening;
an ultraviolet light source and/or a bipolar ionization element mounted within the housing and effective to inactivate virus particles in air passing through the housing in a path from the air intake grille of the housing to the air passage opening of the housing.

2. The vehicle of claim 1, wherein the vehicle is a bus, preferably a school bus.

3. The vehicle of claim 1, wherein the wall is a back wall or front wall bounding the interior passenger compartment.

4-9. (canceled)

10. The vehicle of claim 1, wherein the housing has a hingedly connected panel for providing access to the interior of the housing.

11. The vehicle of claim 10, also comprising a switch electrically connected to an electrical power source for the ultraviolet light or the bipolar ionization element, the switch arranged to interrupt power to the ultraviolet light or the bipolar ionization element when the panel is opened.

12. The vehicle of claim 11, wherein the heating and/or cooling system and the housing are sized and configured to pass air through the housing at a rate of at least 800 feet per minute, preferably in the range of about 800 to about 1500 feet per minute, and more preferably about 1000 to about 1500 feet per minute.

13. The vehicle of claim 12, also including a washable filter mounted in the housing through which the air passes after passage through the air intake grille and before passage by the ultraviolet light source or bipolar ionization element.

14. (canceled)

15. The vehicle of claim 1, wherein the air intake grill prevents direct passage of UV rays from within the housing.

16-19. (canceled)

20. A method for retrofitting a vehicle to provide a retrofitted vehicle capable of viral inactivation, the vehicle having an air conditioned interior passenger compartment, the vehicle having a first air intake opening in a wall through which air is removed from an interior passenger compartment of the vehicle for conditioning by a heating and/or cooling system, and a first air outflow opening through which the air is returned to the interior after conditioning by the heating and/or cooling system, the method comprising;
mounting a housing to the wall, the housing including an air intake grille for communicating with the interior passenger compartment and an air passage opening for communicating with the first air intake opening, the housing being arranged and mounted to the wall so that the air removed from the interior passenger compartment passes through the housing and then through the air intake opening; and
providing an ultraviolet light source and/or a bipolar ionization element mounted within the housing and effective to inactivate virus particles in air passing through the housing in a path from the air intake grille of the housing to the air passage opening of the housing.

21. The method of claim 20, wherein the retrofitted vehicle is a vehicle according to any one of claims 2 to 19.

22. A method for inactivating virus particles in air in an interior passenger compartment of a vehicle, the vehicle having a first air intake opening in a wall through which air is removed from the interior passenger compartment of the vehicle for conditioning by a heating and/or cooling system of the vehicle, and a first air outflow opening through which the air is returned to the interior passenger compartment after conditioning by the heating and/or cooling system, the method comprising;
passing the air which is removed from the interior passenger compartment through a housing mounted to the wall and including an air intake grille communicating with the interior passenger compartment and an air passage opening communicating with the first air intake opening, the housing being arranged so that the air removed from the interior passenger compartment passes through the housing and then through the air intake opening; and
treating the air passing through the housing with an ultraviolet light source and/or a bipolar ionization element mounted within the housing so as to inactivate virus particles.

23-35. (canceled)

36. A kit for retrofitting a vehicle for viral inactivation, the vehicle having an air conditioned interior passenger compartment, the vehicle having a first air intake opening in a wall through which air is removed from the interior passenger compartment for conditioning by a heating and/or cooling system, and a first air outflow opening through which the air is returned to the interior passenger compartment after conditioning by the heating and/or cooling system, the kit comprising:
a housing mountable to the wall and including an air intake grille for communicating with the interior passenger compartment and an air passage opening for communicating with the first air intake opening, the housing being arranged so that when mounted to the wall the air removed from the interior passenger compartment passes through the housing and then through the air intake opening; and
an ultraviolet light source and/or a bipolar ionization element mounted or mountable within the housing and effective to inactivate virus particles in air passing through the housing in a path from the air intake grille of the housing to the air passage opening of the housing.

37-49. (canceled)

50. A vehicle having an onboard air treatment system that inactivates virus particles, comprising:
a vehicle having a passenger compartment, the vehicle having an air return opening through which air is removed from an interior passenger compartment of the vehicle for travel through an air circulation system, and an air supply opening through which the air is supplied to the interior after travel through the air circulation system; and
an ultraviolet light source and/or a bipolar ionization element positioned and effective to inactivate virus particles in the air.

51. The vehicle of claim 50, comprising said ultraviolet light source, and also comprising:
a return grille over the return opening; and
means for blocking ultraviolet radiation positioned between the return grille and the ultraviolet light source, said means for blocking optionally mounted to the return grille.

52-54. (canceled)

55. The vehicle of claim 50, also comprising an electrostatic filter element including a fibrous dielectric filter media to which voltage is applied to polarize the fibrous dielectric filter media, the electrostatic filter element positioned so that the air passes through the fibrous dielectric filter media for filtration.

56. The vehicle of claim 55, wherein the electrostatic filter element has a MERV equivalent rating of MERV-13 equivalent or greater.

57-66. (canceled)

67. An apparatus for installation in a vehicle to treat air of a passenger compartment of the vehicle, comprising:
an ultraviolet light source and/or a bipolar ionization element positionable to inactivate virus particles in the air; and
a mount member mountable to the vehicle and supporting the ultraviolet light source and/or bipolar ionization element.

68. The apparatus of claim 67, comprising said ultraviolet light source, and also comprising:
a return grille for mounting over a return air opening into the passenger compartment; and
means for blocking ultraviolet radiation positionable between the return grille and the ultraviolet light source, said means for blocking optionally mounted to the return grille.

69-71. (canceled)

72. The apparatus of claim 67, also comprising an electrostatic filter element including a fibrous dielectric filter media to which voltage is applied to polarize the fibrous dielectric filter media, the electrostatic filter element positioned so that the air passes through the fibrous dielectric filter media for filtration.

73. The apparatus of claim 72, wherein the electrostatic filter element has a MERV equivalent rating of MERV-13 equivalent or greater.

74-92. (canceled)

93. An apparatus for installation in a vehicle to treat air circulated through a passenger compartment of the vehicle, comprising:
an electrostatic filter element including a dieletric filter media to which voltage is applied to polarize the dielectric filter media, the electrostatic filter element positionable so that the air passes through the dielectric filter media for filtration; and
a frame mountable to the vehicle and supporting the electrostatic filter element.

94-106. (canceled)

107. An apparatus for treating air, comprising:
an electrostatic filter element including a fibrous dielectric filter media to which voltage is applied to polarize the dielectric filter media, the electrostatic filter element positionable so that the air passes through the fibrous dielectric filter media for filtration, the air entering the fibrous dielectric filter media at a first surface thereof and exiting the fibrous dielectric filter media at a second surface thereof;
a secondary fibrous media that is less penetrable to the ultraviolet light than the fibrous dielectric filter media; and
an ultraviolet light source that emits ultraviolet light, the ultraviolet light source positioned and effective to inactivate virus particles in the air and to irradiate the fibrous dielectric filter media with the ultraviolet light in a direction from a the second side of the fibrous dielectric filter media to the first side of the fibrous dielectric filter media; and
wherein the secondary fibrous media is effective to reduce the intensity of the ultraviolet light at a position spaced outwardly from the first surface of the fibrous dielectric media.

108-132. (canceled)

133. A method for treating air of a passenger compartment of a vehicle, comprising:
passing the air through a return grille exposed to the passenger compartment;
after said passing, irradiating the air with ultraviolet light to inactivate viral particles in the air;
blocking the ultraviolet light from entering the passenger compartment with means for blocking ultraviolet light, the means for blocking ultraviolet light associated with the return grille; and
after said irradiating, supplying the air to the passenger compartment through a supply opening into the passenger compartment.

134-139. (canceled)
